(19) United States
(12) Patent Application Publication
(10) Pub. No.: US 2009/0105525 A1
(43) Pub. Date: Apr. 23, 2009

(54) Environment-Affecting Meditation Tool Systems

(76) Inventor: Sanat Maitreya Kumara, Hidden Valley Lake, CA (US)

Correspondence Address: Stoneman Volk Patent Group, 3770 NORTH 7TH STREET, Suite 100, PHOENIX, AZ, 85014, US

(21) Appl. No.: 12/253,784

(22) Filed: Oct. 17, 2008

Related U.S. Application Data
(60) Provisional application No. 60/980,673, filed on Oct. 17, 2007.
(60) Provisional application No. 61/013,964, filed on Dec. 14, 2007.
(60) Provisional application No. 61/045,698, filed on Apr. 17, 2008.

(51) Int. Cl. A61M 21/00

(52) U.S. Cl. 600/27

(57) ABSTRACT

Environment-affecting meditation systems and environment-affecting meditation tools based on combinations of sacred objects. Further, the meditation tools provide for specific arrangements of such tools environments conducive for user meditation. Also, this invention makes available such meditation tools and related spiritual teachings for sale via ecommerce means and by deployment to other industries.

### CROSS-REFERENCE TO RELATED APPLICATIONS

The present application is related to and claims priority from prior provisional patent application Ser. No. 60/980,673, filed Oct. 17, 2007, entitled “MEDITATION & HEALING TOOLS”, and is also related to and claims priority from prior provisional patent application Ser. No. 61/013,964, filed Dec. 14, 2007, entitled “SYSTEMS FOR DEVELOPING AND SELLING SACRED OBJECTS”, and is also related to and claims priority from prior provisional patent application Ser. No. 61/045,698, filed Apr. 17, 2008, entitled “ENVIRONMENT-AFFECTING MEDITATION TOOL SYSTEMS”, the contents of all of which are incorporated herein by this reference and are not admitted to be prior art by reference in this cross-reference section.

### BACKGROUND

This invention relates to environment-affecting meditation tool systems. More particularly, it relates to environment-affecting meditation tools and methods of making, using, and marketing such tools. Even more particularly, this invention relates to such environment-affecting meditation tools made using combinations of objects sacred to meditators. Also, this invention relates to user selection of objects and objects characteristics with which to create custom environment-affecting meditation tools.

Further, this invention relates to specific arrangements of such environment-affecting meditation tools usable in environments conducive to meditation. Further, this invention relates to the making available such tools for sale via electronic commerce (“ecommerce”) systems. Even further, this invention relates to monetizing the time users spend within a meditative environment by licensing others to deploy such environments.

Many “Eastern” practices relating to meditation have been adopted by those in the Western world to assist in relieving stress to heal the mind and body. There is a need for tools useful in assisting meditation. Further, there exists a need to provide those who meditate (meditators) with the means to customize their meditation environment. Also, there is a need for an electronic means for ordering customized environment-affecting meditation tools. Further, there is a need for a method to develop environment-affecting meditation tool, based on combinations of sacred objects, having repeating characteristics sanctioned by a sanctioning entity. Further, there is a need to provide training of others in the use of the environment-affecting meditation tools of the present invention. Still, even further, there exists a need to provide establishments with the means to provide environment-affecting meditation tools for the benefit of the patrons of the establishment.

### OBJECTS AND FEATURES OF THE INVENTION

A primary object and feature of the present invention is to provide a system overcoming the above-mentioned problem and meeting the above-mentioned needs.

It is a further object and feature of the present invention to provide an environment-affecting meditation tool useful to assist meditation. It is a further object and feature of the present invention to provide a system where a user may select objects and objects characteristics such a system comprising methods to monetize the custom ordering and fulfilling to end users of environment-affecting meditation tools, the meditation tool's structure based on combinations of sacred objects, having repetitive characteristics.

A further primary object and feature of the present invention is to provide such a system that is efficient, inexpensive and handy. Other objects and features of this invention will become apparent with reference to the following descriptions.

### SUMMARY OF THE INVENTION

In accordance with a preferred embodiment hereof, this invention provides an environment-affecting meditation tool system comprising at least one bar having at least one terminal end; at least one pair of magnets; and at least one conductive wire; wherein such at least one conductive wire attaches such at least one pair of magnets to such at least one bar by externally wire-wrapping such at least one pair of magnets; and wherein each such magnet comprises a minimum magnetic energy product of about 24 megagauss-oersted; wherein at least one immediate environment of such meditation tool is affected; and wherein at least one particular feature of such environment-affecting meditation tool is custom-selectable by at least one user of such meditation tool from a variety of such custom-selectable features sacred to such user. Moreover, it provides such an environment-affecting meditation tool, wherein such at least one particular feature comprises such at least one conductive wire comprising sterling silver. Additionally, it provides such an environment-affecting meditation tool, wherein such at least one particular feature comprises such at least one conductive wire comprising sterling silver; such sterling silver consists essentially of silver alloy; and such alloy consists essentially of high purity silver. Also, it provides such an environment-affecting meditation tool, wherein such at least one particular feature comprises such at least one conductive wire comprising gold. In addition, it provides such an environment-affecting meditation tool, wherein such at least one particular feature comprises such at least one conductive wire comprising gold; such gold consists essentially of gold alloy; and such gold alloy consists essentially of a minimum of 14 Karat purity.

And, it provides such an environment-affecting meditation tool, wherein such at least one particular feature comprises such at least one conductive wire comprising platinum. Further, it provides such an environment-affecting meditation tool, wherein such at least one particular feature comprises such at least one conductive wire comprising platinum; such platinum consists essentially of platinum alloy; and such platinum alloy consists essentially of high purity platinum. Even further, it provides such an environment-affecting meditation tool, wherein such at least one particular feature comprises such at least one conductive wire comprising titanium. Moreover, it provides such an environment-affecting meditation tool, wherein such at least one particular feature comprises such at least one conductive wire comprising titanium; such titanium consists essentially of titanium alloy; and such titanium alloy consists essentially of high purity titanium. Additionally, it provides such an environment-affecting meditation tool, wherein such at least one particular feature comprises; such at least one bar comprising crystal; wherein such crystal consists essentially of quartz; and wherein such quartz consists essentially of Siberian quartz. Also, it provides such an environment-affecting meditation tool, wherein such at least one particular feature comprises at least one indicia; wherein such at least one indicia comprises an etching; and wherein such at least one indicia is on such at least one terminal end of such at least one bar.

In addition, it provides such an environment-affecting meditation tool, wherein such at least one particular feature comprises at least one extender extending outwardly from adjacent such at least one terminal end of such at least one bar; wherein such at least one extender comprises at least one chain and at least one handle; and at least one split-ring configuration connecting such at least one chain to such at least one handle. And, it provides such an environment-affecting meditation tool, wherein such at least one particular feature comprises such at least one extender comprises at least one geometric configuration; wherein such at least one geometric configuration is attached with such at least one extender adjacent such at least one terminal end of such at least one bar; and wherein such at least one chain is attached to such at least one geometric configuration attached with such at least one extender. Further, it provides such an environment-affecting meditation tool, further comprising at least one framework; at least one gemstone; and at least one disc. Even further, it provides such an environment-affecting meditation tool, wherein such at least one particular feature comprises such at least one framework comprising gold. Moreover, it provides such an environment-affecting meditation tool, wherein such gold consists essentially of gold alloy; and such gold alloy consists essentially of a minimum of 14-Karat purity. Additionally, it provides such an environment-affecting meditation tool, wherein such at least one particular feature comprises such at least one framework comprising titanium. Also, it provides such an environment-affecting meditation tool, wherein such titanium consists essentially of titanium alloy; and such titanium alloy consists essentially of high purity titanium. In addition, it provides such an environment-affecting meditation tool, wherein such at least one particular feature comprises such at least one framework comprising sterling silver. And, it provides such an environment-affecting meditation tool, wherein such sterling silver consists essentially of silver alloy; and such silver alloy consists essentially of high purity silver.

Further, it provides such an environment-affecting meditation tool, wherein such at least one particular feature comprises such at least one framework comprising platinum. Even further, it provides such an environment-affecting meditation tool, wherein such platinum consists essentially of platinum alloy; and such platinum alloy consists essentially of high purity platinum. Moreover, it provides such an environment-affecting meditation tool, wherein such at least one particular feature comprises at least four of such at least one bar, each bar having at least one terminal end; wherein such at least one framework supports such at least four bars. Additionally, it provides such an environment-affecting meditation tool, wherein such at least one particular feature comprises such at least one gemstone comprising turquoise. Also, it provides such an environment-affecting meditation tool, wherein such at least one particular feature comprises such at least one gemstone comprising diamond. In addition, it provides such an environment-affecting meditation tool, wherein such diamond consists essentially of one-half carat total weight. And, it provides such an environment-affecting meditation tool, wherein such at least one particular feature comprises such at least one gemstone comprising sapphire. Further, it provides such an environment-affecting meditation tool, wherein such at least one particular feature comprises such at least one gemstone comprising ruby. Even further, it provides such an environment-affecting meditation tool, wherein such at least one particular feature comprises such at least one gemstone comprising topaz. Moreover, it provides such an environment-affecting meditation tool, wherein such at least one particular feature comprises such at least one gemstone comprising crystal.

Additionally, it provides such an environment-affecting meditation tool, wherein such crystal consists essentially of quartz; wherein such quartz consists essentially of Siberian quartz. Also, it provides such an environment-affecting meditation tool, wherein such at least one particular feature comprises at least one setting comprising at least four prongs; wherein such at least four prongs are positioned substantially so as not to be congruous with such at least one terminal end of such at least one bar; wherein such at least four prongs secure such at least one gemstone on such at least one framework; and wherein such at least one setting is configured such that such at least one gemstone will not be at a elevation higher than such at least one framework. In addition, it provides such an environment-affecting meditation tool, wherein such at least one particular feature comprises such at least one disc comprising crystal; wherein such disc-crystal consists essentially of quartz; and wherein such disc-crystal-quartz consists essentially of Siberian quartz.

In accordance with another preferred embodiment hereof, this invention provides an environment-affecting meditation tool system comprising at least one bar having at least one terminal end; at least one pair of magnets; and at least one conductive wire; wherein such at least one conductive wire attaches such at least one pair of magnets to such at least one bar by externally wire-wrapping such at least one pair of magnets; and wherein each of such at least one pair of magnets comprises a minimum energy product of about 24 megagauss-oersted; wherein at least one immediate environment of such meditation tool is affected; and wherein such at least one conductive wire of such environment-affecting meditation tool comprises gold.

In accordance with another preferred embodiment hereof, this invention provides an environment-affecting meditation tool system comprising at least one bar having at least one terminal end; at least one pair of magnets; and at least one conductive wire; wherein such at least one conductive wire attaches such at least one pair of magnets to such at least one bar by externally wire-wrapping such at least one pair of magnets; and wherein each of such at least one pair of magnets comprises a minimum energy product of about 24 megagauss-oersted; wherein at least one immediate environment of such meditation tool is affected; and wherein such at least one conductive wire of such environment-affecting meditation tool comprises silver.

In accordance with another preferred embodiment hereof, this invention provides an environment-affecting meditation tool system comprising at least one bar having at least one terminal end; at least one pair of magnets; and at least one conductive wire; wherein such at least one conductive wire attaches such at least one pair of magnets to such at least one bar by externally wire-wrapping such at least one pair of magnets; and wherein each of such at least one pair of magnets comprises a minimum energy product of about 24 megagauss-oersted; wherein at least one immediate environment of such meditation tool is affected; and wherein such at least one conductive wire of such environment-affecting meditation tool comprises platinum.

In accordance with another preferred embodiment hereof, this invention provides an environment-affecting meditation tool system comprising at least one bar having at least one terminal end; at least one pair of magnets; and at least one conductive wire; wherein such at least one conductive wire attaches such at least one pair of magnets to such at least one bar by externally wire-wrapping such at least one pair of magnets; and wherein each of such at least one pair of magnets comprises a minimum energy product of about 24 megagauss-oersted; wherein at least one immediate environment of such meditation tool is affected; and wherein such at least one conductive wire of such environment-affecting meditation tool comprises titanium.

In accordance with another preferred embodiment hereof, this invention provides an environment-affecting meditation tool system comprising at least one bar having at least one terminal end; at least one pair of magnets; at least one conductive wire; and at least one fragment of sacred stone; wherein such at least one fragment of sacred stone is attached to such at least one bar; wherein such at least one conductive wire attaches such at least one pair of magnets to such at least one bar by externally wire-wrapping such at least one pair of magnets; wherein each of such at least one pair of magnets comprises a minimum energy product of about 24 megagauss-oersted; wherein at least one immediate environment of such meditation tool is affected; and wherein such at least one conductive wire of such environment-affecting meditation tool comprises titanium; and wherein such at least one fragment of sacred stone comprises moldivite.

In accordance with another preferred embodiment hereof, this invention provides a method, relating to environment-affecting meditation tools, comprising the steps of sanctioning as sacred, by at least one church group, at least one first set of objects; wherein the at least one first set of sacred objects consists essentially of environment-affecting objects; and sanctioning, by the at least one church group, use of the at least one first set of environment-affecting sacred objects to at least assist meditation; and sanctioning, by the at least one church group, specified combining of at least one second set of objects with at least one of the first set of environment-affecting sacred objects to create at least one third set of sacred objects, the third set of sacred objects comprising a large variation of environment-affecting sacred meditation tools; and permitting, by the at least one church group, respective purchasers of the at least one third set of sacred objects to respectively custom design at least one respective custom object, by use of at least one designated website of the at least one church group. And, it provides such a method, further comprising the steps of providing receiving payment for the at least one respective custom object; providing making, once sanctioned by the at least one church group, of the at least one respective custom object; and providing fulfilling of the at least one respective custom object to the respective purchaser of such at least one respective custom object. Further, it provides such a method, further comprising sanctioning, by the at least one church group, training at least one other to provide church-group-sanctioned meditation services within at least one space, the space structured and arranged as specified by the at least one church group, the church-group-sanctioned meditation services using at least one of the first set of sacred objects consisting essentially of environment-affecting objects.

Even further, it provides such a method, wherein such at least one other so trained comprise at least one spa service provider. Even further, it provides such a method, further comprising providing, to such at least one trained spa service provider having at least one site, on-site retail distribution rights to sell the environment-affecting sacred meditation tools. Even further, it provides such a method, further comprising the steps of providing sanctioning, by the at least one church group, of the at least one respective custom object; providing at least one customer purchasing interface for selecting at least one custom aspect for such at least one respective custom object and purchasing the at least one respective custom object; providing at least one merchant-banking payment system so that the at least one customer can provide payment for such at least one respective custom object; providing at least one assembling tracking system to track assembly of the at least one respective custom object; and providing at least one fulfillment tracking system to track fulfillment of the purchase.

In accordance with another preferred embodiment hereof, this invention provides a computer-implemented method comprising the steps of providing at least one first customer interface, accessible over at least one computer network, wherein a customer may select at least one environment-affecting meditation-tool to be purchased; providing at least one second customer interface, accessible over at least one computer network, wherein a customer may select at least one particular custom feature of the selected environment-affecting meditation-tool to be purchased; processing, by at least one processor, custom input selections of the at least one customer; and outputting at least one custom specification listing of the custom environment-affecting meditation-tool selected to be purchased by the at least one customer; and storing such custom specifications of the custom environment-affecting meditation-tool. Even further, it provides such a computer-implemented method, further comprising the steps of providing at least one customer purchase interface, accessible over at least one computer network, wherein a customer may input purchase-related information; providing at least one purchasing processor to carry out a customer purchase transaction using such customer input purchase-related information; outputting display of confirmation of a purchase to the at least one purchasing customer.

Even further, it provides such a computer-implemented method, further comprising the step of providing at least one purchase fulfillment tracking processor to track fulfillment-related data of a purchased at least one custom environment-affecting meditation tool. Even further, it provides such a computer-implemented method, further comprising the step of providing at least one assembly fulfillment tracking processor to track assembly-related data of at least one custom environment-affecting meditation tool. Even further, it provides such a computer-implemented method, further comprising the step of providing at least one custom environment-affecting meditation-tool delivery fulfillment tracking processor to track delivery-related data relating to a purchased at least one custom environment-affecting meditation tool. Even further, it provides such a computer-implemented method, further comprising providing at least one real-time customer-messaging interface, accessible over at least one computer network, to permit real-time input of a customer message; and providing at least one customer messaging processor to process the customer message to be displayed on at least one other computer connected to the at least one computer network.

### BRIEF DESCRIPTION OF THE DRAWINGS

FIG. 1 shows a high-level schematic diagram, illustrating preferred business architectures relating to environment-affecting meditation tools, according to a preferred embodiment of the present invention.

FIG. 2 shows a more detailed schematic diagram, illustrating preferred business architectures relating to environment-affecting meditation tools, according to the preferred embodiment of FIG. 1.

FIG. 3 shows a detailed schematic diagram, illustrating a preferred implementation of the business architecture relating to environment-affecting meditation tools according to the preferred embodiments of FIG. 1 and FIG. 2.

FIG. 4 shows an illustration of a screenshot illustrating the ability of a customer to selectively customize an environment-affecting meditation tool according to the preferred embodiment of the present invention.

FIG. 5 shows another illustration of a screenshot illustrating the ability of a customer to selectively customize an environment-affecting meditation tool according to the preferred embodiment of the present invention.

FIG. 6 shows a schematic diagram view, of typical hardware to implement a system for offering customizing of environment-affecting meditation tools according to the preferred embodiment of the present invention.

FIG. 7 shows a flowchart illustrating a decision tree involved with sanctioning requested configurations of environment-affecting tools.

FIG. 8 shows a simplified schematic diagram illustrating a method of combining environment-affecting meditation tools according to a preferred embodiment of the present invention.

FIG. 9 shows several customer-selectable options of sacred objects and materials.

FIG. 10 shows a perspective view illustrating a first environment-affecting meditation tool form of the environment-affecting meditation tool system according to a preferred embodiment of the invention.

FIG. 11 shows a perspective view illustrating a second environment-affecting meditation tool form of the environment-affecting meditation tool system according to a preferred embodiment of the invention.

FIG. 12 shows a front plan view illustrating a third environment-affecting meditation tool form of the environment-affecting meditation tool system according to a preferred embodiment of the invention.

FIG. 13 shows a back plan view of the embodiment of FIG. 12.

FIG. 14 shows a perspective view illustrating a fourth environment-affecting meditation tool form of the environment-affecting meditation tool system according to a preferred embodiment of the invention.

FIG. 15 shows a perspective view illustrating a fifth environment-affecting meditation tool form of the environment-affecting meditation tool system according to a preferred embodiment of the invention.

FIG. 16 shows a front view illustrating a sixth environment-affecting meditation tool form of the environment-affecting meditation tool system according to a preferred embodiment of the invention.

FIG. 17 shows a front perspective view illustrating a seventh environment-affecting meditation tool form of the environment-affecting meditation tool system according to a preferred embodiment of the invention.

FIG. 18 shows an exploded view illustrating an eight environment-affecting meditation tool form of the environment-affecting meditation tool system according to a preferred embodiment of the invention.

FIG. 19 shows a close up view of a middle layer of the embodiment of FIG. 18.

FIG. 20 shows additional components of the embodiment of FIG. 18.

FIG. 21 shows a perspective view illustrating a ninth environment-affecting meditation tool form of the environment-affecting meditation tool system according to a preferred embodiment of the invention.

FIG. 22 shows a perspective view illustrating a corner piece system of the embodiment of FIG. 21.

FIG. 23 shows a schematic diagram illustrating a preferred method of the present invention.

### DETAILED DESCRIPTION OF THE BEST MODES AND PREFERRED EMBODIMENTS OF THE INVENTION

This detailed description will describe the following areas of the environment-affecting meditation tool systems of the present invention: (a) business architectures and associated methods relating to the communicating, fulfilling, ordering, customizing, selling, etc., of environment-affecting meditation tools, (b) preferred structures and features of environment-affecting meditation tools, and (c) deployment to customers of particular environment-affecting meditation tools and services related to the use of such particular environment-affecting meditation tools.

### PART I

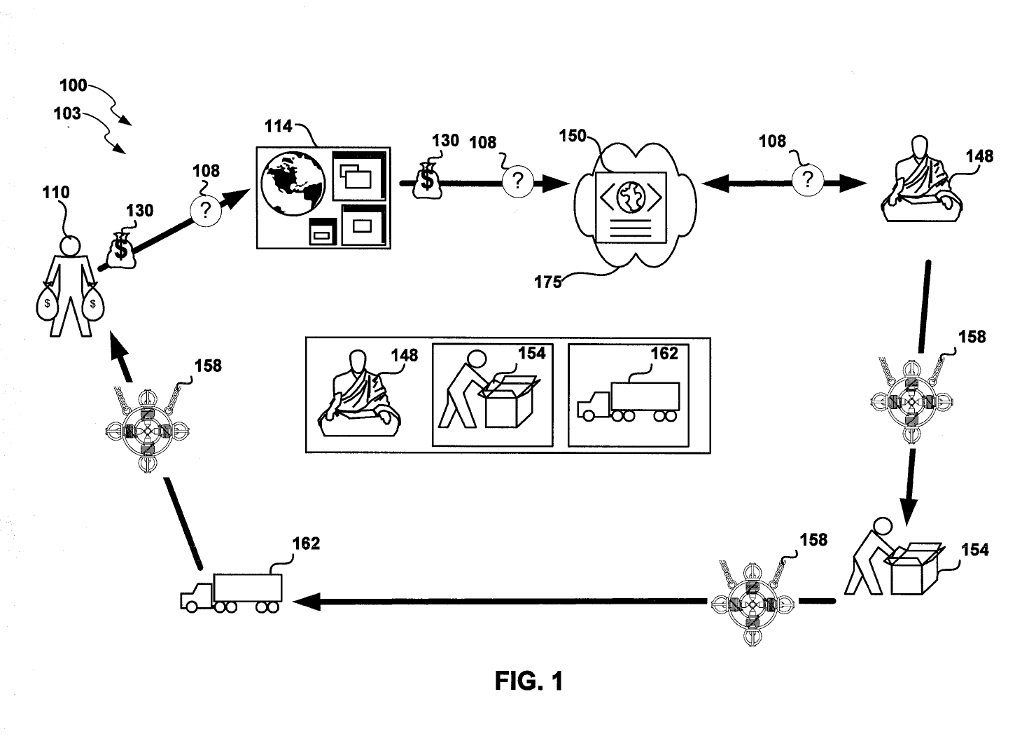

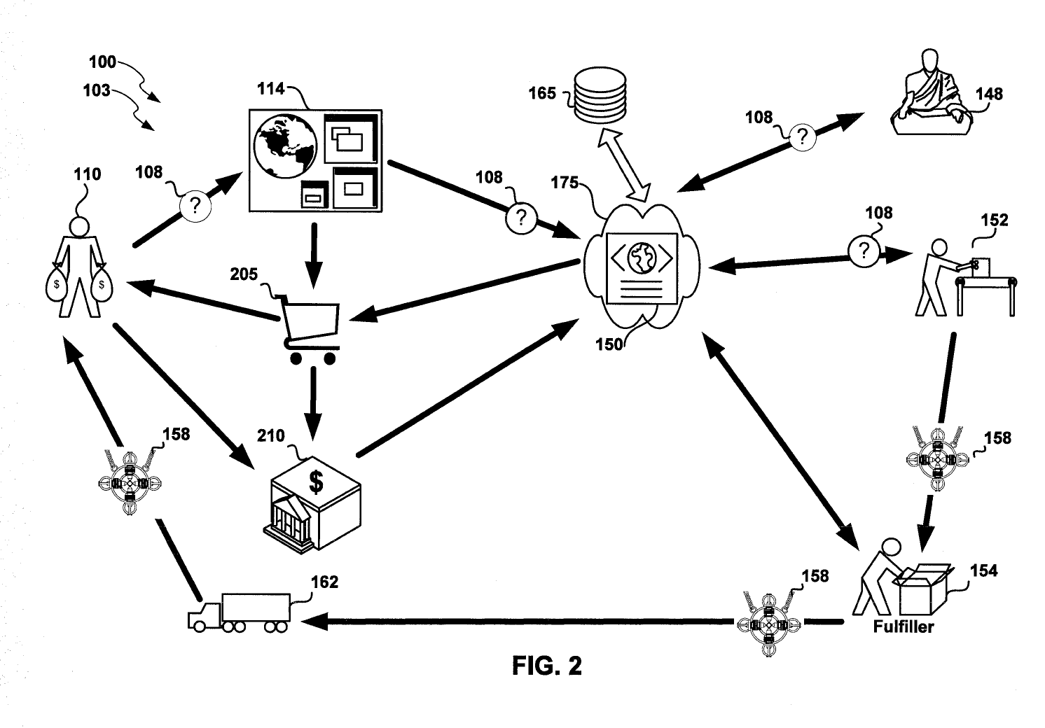

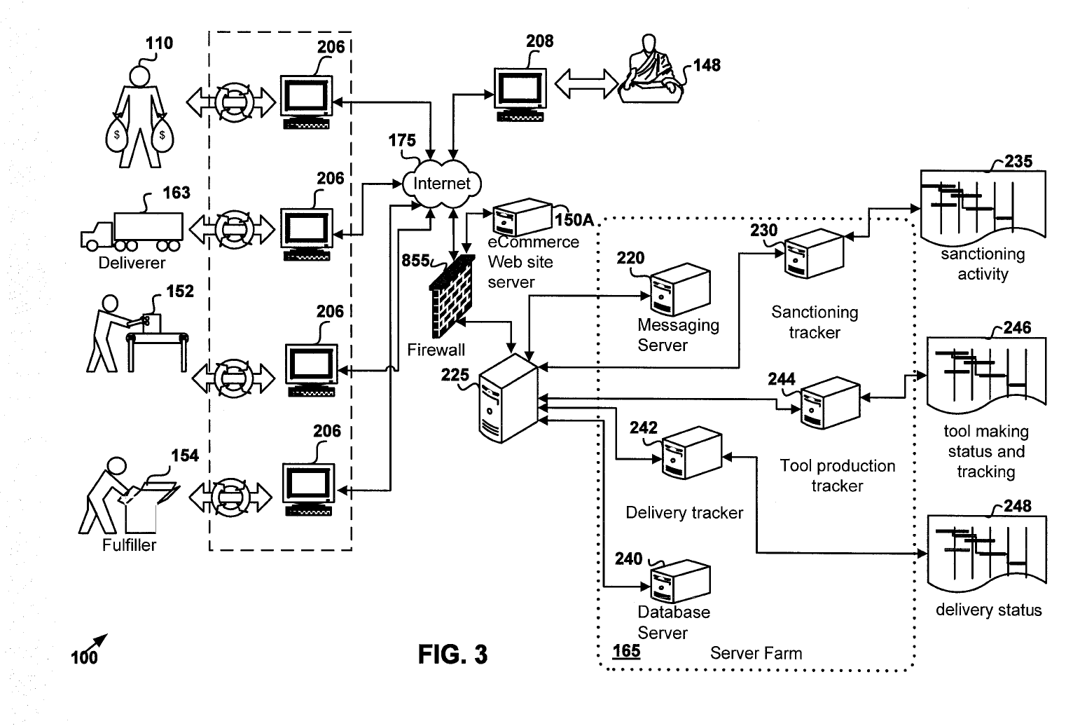

Environment-affecting meditation tool system 100 preferably comprises method 103, as shown. Method 103 involves the convergence and working together of entities that do not conventionally deal with one another, as described herein. A preferred central entity, or hub, of environment-affecting meditation tool systems 100, and of method 103, is, preferably, sanctioning church entity 148, as shown in FIG. 1, FIG. 2, and FIG. 3. The term “church” is used herein to refer to a collective group that follows a set of guidelines common to the collective group. Sanctioning church entity 148 preferably comprises a close group of church members that assist, preferably, a single individual in carrying out church-related activities and missions. Upon reading the teachings of this specification, those of ordinary skill in the art will now understand that, under appropriate circumstances, considering such issues as number of church members, church government, church politics, etc., other arrangements for sanctioning church entity, such as a group of church followers, an elected group of church members, a rotating group of church members, outside church advisors, etc., may suffice.

Sanctioning church entity 148 preferably plays many roles in method 103. Preferably, sanctioning church 148 may preferably act as system developer to design, implement, and manage ecommerce web site 150. Also preferably sanctioning church 148 may preferably act as teaching entity. As a teaching entity, the role of sanctioning church entity 148 is to interface with customers 110 (and generally interface with users of environment-affecting meditation tools) to teach and deploy meditative uses of environment-affecting meditation tools, such as environment-affecting tool 158. Sanctioning church 148 may also preferably act to oversee the making of environment-affecting meditation tools 158 and the fulfillment (including sourcing raw materials, packaging, shipping, receiving payment, etc.) of environment-affecting meditation tools 158 to customers 110 when customers 110 wish to purchase and use environment-affecting meditation tools 158, as shown. Finally, sanctioning church 148 may also preferably interact directly with customers 110 to teach customers 110 (who have purchased environment-affecting meditative tools 158) proper meditative use of environment-affecting meditative tools 158. To put method 103 into effect, communication links, preferably ecommerce communication links (preferably Internet 175, as shown) and other links, are preferably established as will be discussed herein in more detail.

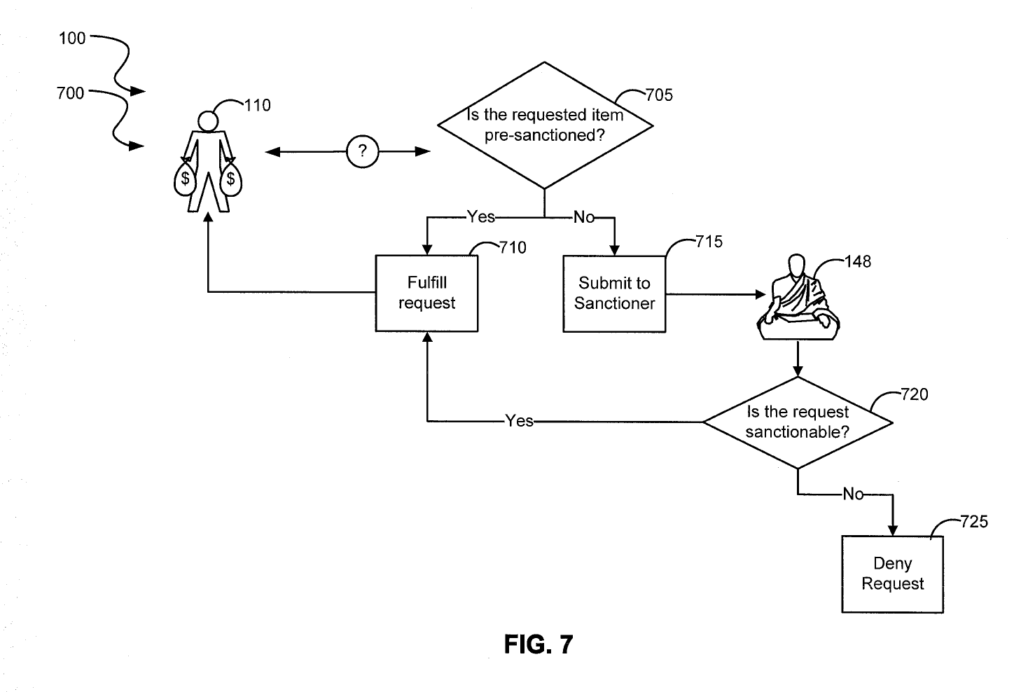

FIG. 1 shows a high-level schematic diagram, illustrating preferred business architectures relating to environment-affecting meditation tools, according to a preferred embodiment of the present invention. As depicted in FIG. 1, environment-affecting meditation tool systems 100 preferably comprise method 103, as shown. With brief reference to FIG. 7, method 103 is preferably implemented to monetize both pre-sanctioned environment-affecting meditation tools as well as sanctionable environment-affecting meditation tools requested by customer 110. Several components must be put into operation so that method 103 will operate effectively.

In one preferred step of method 103, preferably sanctioning church 148 (or also preferably its for-a-fee licensees) preferably arranges designing, implementing, and managing ecommerce web site 150, as shown. The step of arranging designing and implementing ecommerce web site 150 comprises the steps of making arrangements to host ecommerce web site 150 on an ecommerce web site hosting service, publishing ecommerce web site 150 so that ecommerce web site is accessible via a computer network, preferably Internet 175, and managing and maintaining ecommerce web site 150 over time. Typically, a fee is paid to a web site hosting service to host ecommerce web site on Internet 175, as shown.

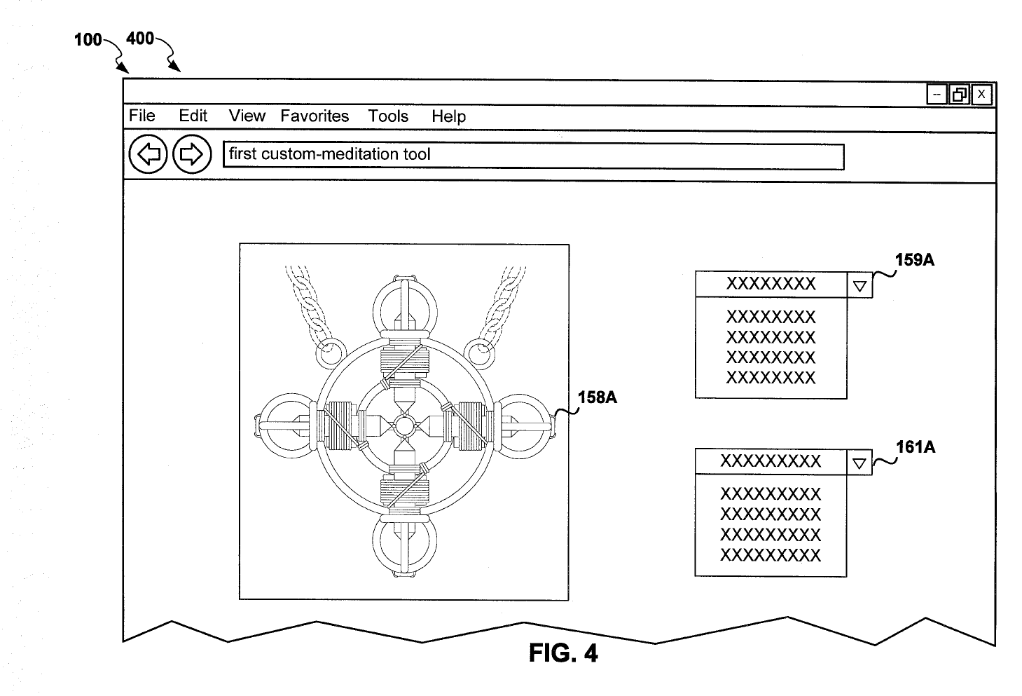

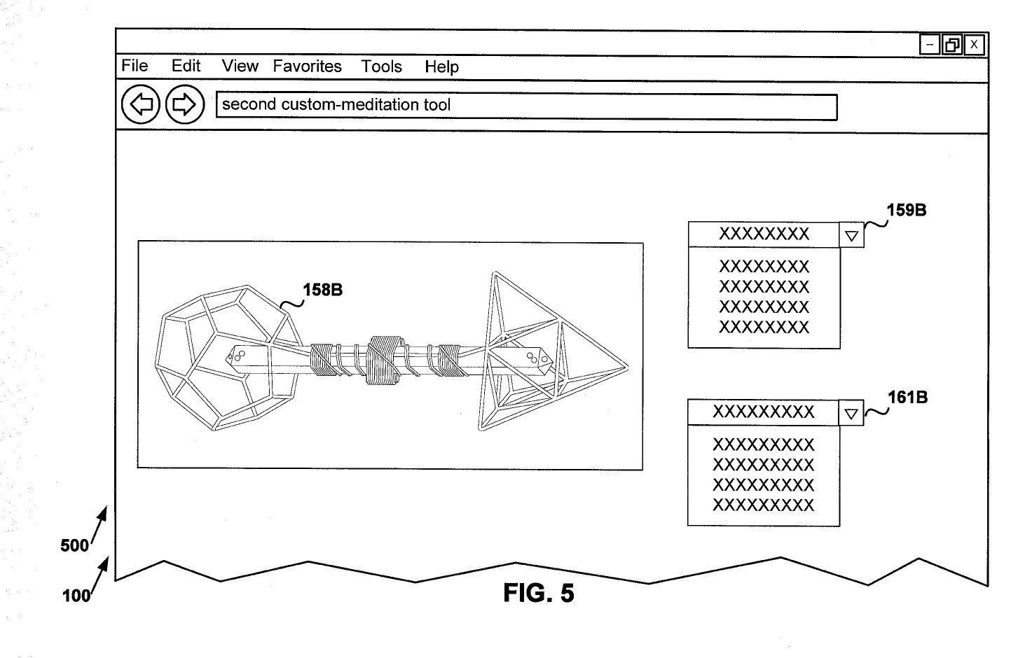

In method 103, customer 110 requests delivery of environment-affecting meditation tool 158 to customer 110, as shown. After web publishing, ecommerce web site 150, preferably comprising customer interface 114, is preferably made available on at least one computer network, preferably Internet 175, as shown. Customer 110 may preferably use customer interface 140 (accessed with a computer communicatively coupled to the computer network) and input order query 108 relating to environment-affecting meditation tools 158 into customer interface 114 of ecommerce web site 150 (see FIG. 4 and FIG. 5).

Using customer interface 114, customer 110 may preferably view and preferably select a pre-sanctioned environment-affecting meditation tool 158 (see FIG. 4 and FIG. 5). Also, customer 110 is also preferably provided with the ability to custom-select at least one particular feature of said environment-affecting meditation tool from a variety of such custom-selectable features sacred to sanctioning church 148 and customer 110 (see FIG. 4 and FIG. 5).

In the event that customer 110 wishes an environment-affecting meditation tool 158 that is not offered by sanctioning church entity 148 on ecommerce web site 150, then such a request is preferably communicated to sanctioning church entity 148 preferably over the Internet, with electronic mail being at least one preferred communication mode. With reference to FIG. 7, if an environment-affecting meditation tool is pre-sanctioned by sanctioning church entity 148, such pre-sanctioned environment-affecting meditation tool will be made available on ecommerce web site 150. Order requests by customers 110 are preferably fulfilled. If customer 110 requests an environment-affecting meditation tool that is not offered on ecommerce web site 150, then the following preferred steps are carried out as preferred method 700 (see FIG. 7). Environment-affecting meditation tool system 100 preferably comprises method 700, as shown. In first preferred step 705, sanctioning church entity 148 determines whether or not the requested configuration is pre-sanctioned. If pre-sanctioned, the request is fulfilled in preferred step 710, as shown. If not pre-sanctioned, the request is preferably submitted, preferably by electronic communication means (preferably electronic mail), to sanctioning church entity 148 in preferred step 715, as shown. Upon reading the teachings of this specification, those of ordinary skill in the art will now understand that, under appropriate circumstances, other communication means, such as instant messaging via an Internet web site, short message service (SMS) messaging, bulletin board posting, newsgroups, etc., may suffice.

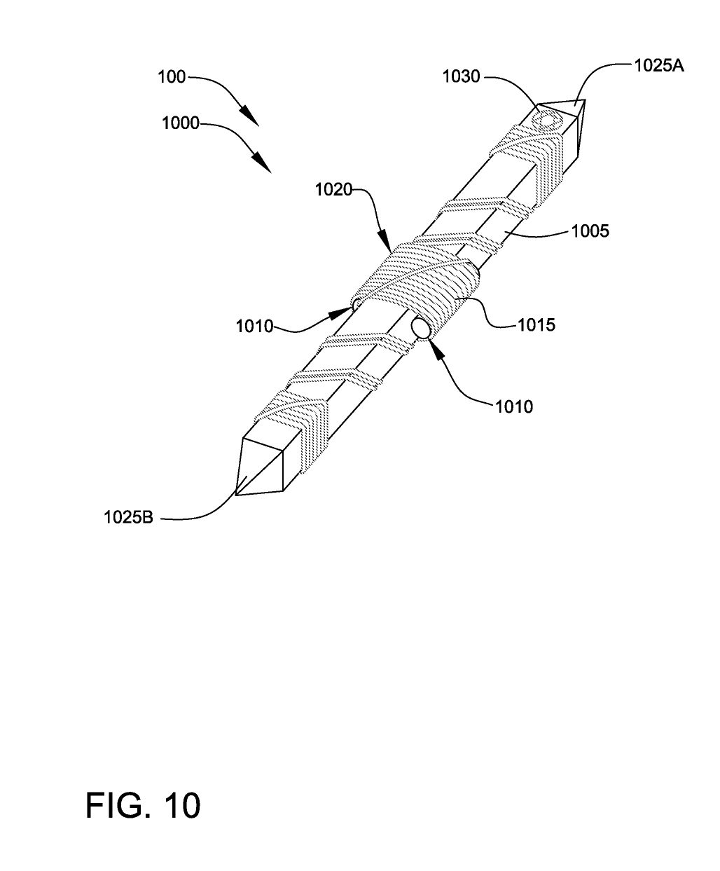

If the requested environment-affecting meditation tool is not pre-sanctioned, sanctioning church entity 148 preferably determines whether the requested item aligns or complies with sanctioning policies of the sanctioning church entity 148 in preferred step 715 (submit request to sanctioner), as shown. If sanctioning church entity 148 determines that the request is sanctionable and complies with such sanctioning policies in preferred step 720, then request fulfillment step 710 is preferably carried out. If, however, sanctioning church entity 148 determines that the request is not sanctionable or does not comply with such sanctioning policies in preferred step 720, the request is denied in preferred step 725, as shown. In a highly preferred embodiment, order query 108 will be sanctionable if they at least comprise at least the item illustrated in FIG. 10 (sacred weaver form 1000). Upon reading the teachings of this specification, those of ordinary skill in the art will now understand that, under appropriate circumstances, considering such issues as materials available, cost of materials, other sanctionable items, such as the item depicted in FIG. 10, objects having particular preferred geometries, octahedrons, tetrahedrons, cubes, star tetrahedrons, dodecahedrons, icosahedrons, combinations of at least two of octahedrons, tetrahedrons, cubes, star tetrahedrons, dodecahedrons, icosahedrons, etc., may suffice.

Returning to FIG. 1 and with respect to initiating fulfillment of order query 108, sanctioning church entity 148 preferably releases environment-affecting meditation tool 158 to fulfiller 154, as shown. Fulfiller 154 is preferably a related business extension of sanctioning church entity 148, as shown. Preferably, fulfiller 154 may deliver environment-affecting meditation tool 158 to distributor 162, as shown. Distributor 162 is preferably a related business extension of sanctioning church entity 148, as shown. Distributor 162 subsequently delivers environment-affecting meditation tool 158 to customer 110, as shown. In exchange for fulfillment of environment-affecting meditation tool 158, customer 110 preferably pays fee 130 preferably using customer interface 114 of ecommerce web site 150, as shown. Preferably, fee 130 payment is handled and processed by ecommerce web site 150 and its related sub-systems (computer processors, input devices, and output devices).

FIG. 2 shows a more detailed schematic diagram, illustrating preferred business architectures relating to environment-affecting meditation tools, according to the preferred embodiment of FIG. 1. Preferably using the Internet, customer 110 may input order query 108 (for at least one environment-affecting meditation tool 158) which is preferably electronically stored within a computer network system, illustrated as database 165. At about the same time that order query 108 is input, customer 110 may also preferably input personal information, billing information, and fulfillment information. Such information is also preferably stored on database 165, as shown. Database 165 is preferably accessible via Internet 175, as shown. Database 165 is also sometimes referred to herein as server farm 165. An order query stored on database 165 may then be preferably communicatively delivered to sanctioning church entity 148, meditation tool maker 152, and fulfiller 154.

Additionally, referring to FIG. 2, ecommerce web site 150 preferably permits communicative delivery of order, design, manufacturing, and fulfillment data to database 165 and preferably receives related reports from database 165, as shown.

Customer 110 preferably submits order query 108 via customer interface 114 of ecommerce web site 150, as shown. Order query 108 is preferably communicated to sanctioning church entity 148, tool maker 152, fulfiller 154, and distributor 162, as shown. Customer 110 preferably orders environment-affecting meditation tool 158 using shopping cart feature 205 of ecommerce web site 150, as shown. Ecommerce web site 150 preferably comprises appropriate communicative links (preferably using Internet 175) to merchant bank 210 to assist transferring payment from customer 110 to church sanctioning entity 148, as shown.

FIG. 3 shows a detailed schematic diagram, illustrating a preferred implementation of the business architecture relating to environment-affecting meditation tools according to the preferred embodiments of FIG. 1 and FIG. 2. Customer 110 preferably interfaces with computer 206 to access ecommerce web site 150, preferably stored and hosted on ecommerce web site server 150A, via Internet 175, as shown. Preferably, once customer 110 connects to ecommerce web site 150, customer 110 has access to the functionality of ecommerce web site 150. For example, customer has access to messaging server 220 to communicate with live attendants/members of sanctioning church entity 148. A preferred software tool providing messaging over an ecommerce web site is the VOLUSION® software package made available by Volusion, Inc., of Simi Valley, Calif. Preferably, customer 110 also has access to delivery tracker server 242, and database server 240, as shown, in database 165.

Preferably environment-affecting meditation tool maker 152, fulfiller 154, and deliverer 163 each preferably interface with computer 206 to access authorized functions of server farm 165, as shown.

Further, FIG. 3 depicts how sanctioning church entity 148 utilizes computer system 208 to enable sanctioning church entity 148 to connect with ecommerce server 150 via Internet 175. Preferably, once sanctioning church entity 148 connects to ecommerce server 150, sanctioning church entity 148 may preferably access the functionality of messaging server 220, delivery tracker 242, sanctioning tracker 230, tool making tracker server 244 and database server 240, as shown, in server farm 165. Additionally, preferably, this connectivity also allows sanctioning church entity 148 to view sanctioning activity 235, tool making status and tracking 246, and tool delivery status 248.

FIG. 4 shows an illustration of a screenshot illustrating the ability of a customer to selectively customize an environment-affecting meditation tool according to the preferred embodiment of the present invention. Preferably, ecommerce ordering page 400 displays at least one image of environment-affecting custom meditation tool 158A, as shown. When customer 110 visits ecommerce ordering page 400, customer 110 is presented with the ability to selectively customize, via customer input, aspects of meditation tool 158A, as shown. Preferably, customer 110 may preferably input desired information using first customizing element 159A and second customizing element 161A. Selections made by customer 110 are stored in database 165 for either making or fulfillment.

FIG. 5 shows another illustration of a screenshot illustrating the ability of a customer to selectively customize an environment-affecting meditation tool according to the preferred embodiment of the present invention. Preferably, ecommerce ordering page 500 comprises second custom meditation tool 158B accompanied by first customizing element 159B and second customizing element 161B, as shown. Preferably, customer 110 uses ecommerce ordering page to input order query 108 and order at least one meditation tool 158B. As discussed above, order query 108 is communicatively transmitted from customer to sanctioning church entity 148, tool maker 152, fulfiller, and deliverer 163, from server farm 165. Further preferably, customer 110 can select at least one first customizing element 159B and at least one second customizing element 161B, when selecting at least one second meditation tool 158B. Still further, preferably, customer 110 can order, through input order query 108, at least one second meditation tool 158B with at least one least one first customizing element 159B and second customizing element 161B.

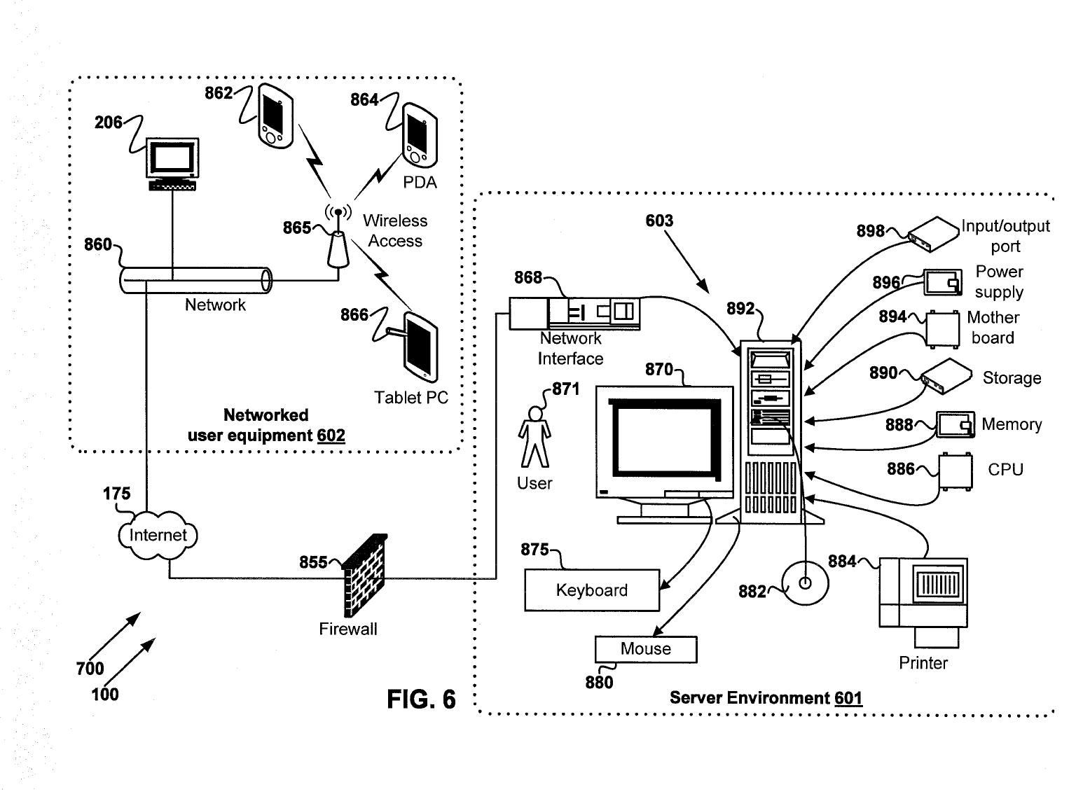

FIG. 6 shows a schematic diagram view, of typical hardware to implement a system for offering customizing of environment-affecting meditation tools according to the preferred embodiment of the present invention. Environment-affecting meditation tool system 100 preferably comprises network environment 700, as shown. Preferably, computer hardware environment 700 comprises server environment 601 and networked user equipment 602, as shown. Additionally, preferably as shown, server environment 601 preferably comprises at least one server 603, as shown. Server 603 preferably comprises at least one processor (such as central processing unit (“CPU”) 886), at least one input device (such as keyboard 875), at least one output device (such as monitor 870), and at least one storage device (such as memory 888). More particularly, server 603 preferably comprises at least one input/output port 898, power supply 896, motherboard 894, chassis 892, storage 890, memory 888, central processing unit (“CPU”) 886, printer 884, removable storage medium 882, mouse 880, keyboard 875, monitor 870, network interface 868, and firewall 855.

Networked user equipment 602 preferably comprises at least one user device, which may be selected from the following: computer 206 (preferably a desktop computer, also preferably a laptop computer); web enabled device 892; personal digital assistant (“PDA”) 894; and tablet personal computer 896. Upon reading the teachings of this specification, those with ordinary skill in the art will now understand that, under appropriate circumstances, considering issues such as changes in technology, user requirements, etc., other monetizing service oriented architectures of computer hardware environment, such as, for example, peer-to-peer network architectures, billing and settlement networking architectures yet to be developed, etc., may suffice.

Preferably chassis 892 of server environment 603, as shown, houses all components that make up server environment 603 and provides at least one input/output port 898. Preferred motherboard 894 of server environment 601 comprises the main logic circuitry and preferably, as shown, provides all necessary circuitry to interconnect all server environment 601 components. Upon reading the teachings of this specification, those with ordinary skill in the art will now understand that, under appropriate circumstances, considering issues such as changes in computer server environment technology, etc., other computer server subassembly and component constructs including elements, such as, for example, highly-integrated motherboards, blade-computer housings yet to be developed, etc., may suffice.

Preferably storage 890 of server environment 601 is preferably configured as a Redundant Array of Independent Drives (RAID) preferably having mirroring (copying of data to more than one physical disk); preferably having striping (splitting of data across more than one physical disk), and preferably error correction or preferably fault tolerance (redundant storing of data that allows detecting and fixing of data-integrity anomalies).

Preferably memory 888 of server environment 601 includes dynamic RAM (random access memory), preferably semiconductor chips that store system software, programs, and data currently used, as shown, by CPU (central processor unit) 886. Memory 888 comprises volatile memory, that is, memory contents of which may be lost when/if server environment 601 loses power. Thus, contents of memory 888 preferably must be stored in storage 890 prior to powering down hardware environment 700.

Preferably CPU (central processing unit) 886 of server environment 601 interprets and executes actual computing tasks of system 100 and preferably has capacity for simultaneously executing multiple processing threads (parallel processing).

Preferably network interface 868 enables preferred server environment 601 to send at least one communication signal and to receive at least one communication signal from network 860, preferably using at least one standards-based communication-protocol.

FIG. 7 shows a flowchart illustrating a decision tree involved with sanctioning requested configurations of environment-affecting tools.

### PART II

The following discussion will turn to preferred structures and features of environment-affecting meditation tools of environment-affecting meditation tool system 100.

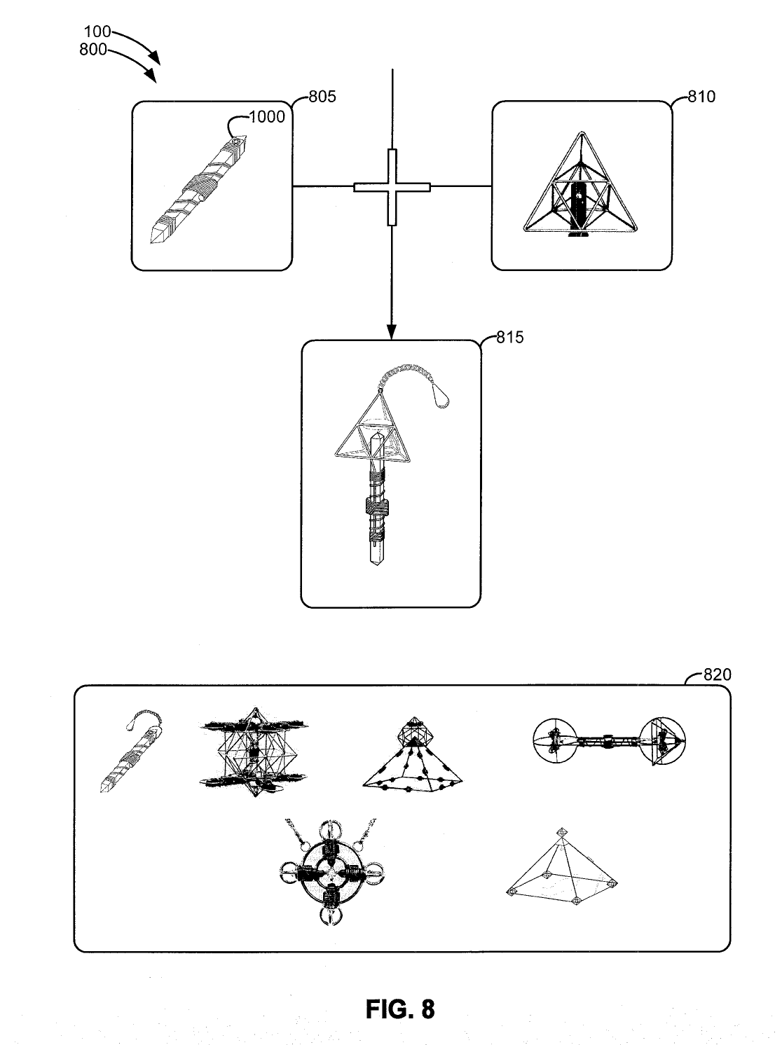

FIG. 8 shows a simplified schematic diagram illustrating a method of combining environment-affecting meditation tools according to a preferred embodiment of the present invention. Environment-affecting meditation tool system 100 preferably comprises method 800, as shown. The embodiments described below preferably derive from basic sacred form 805, as shown. Basic sacred form 805 is preferably sacred weaver form 1000, as shown. Sacred weaver form 1000 is further described in more detail below. According to method 800, additional embodiments 815 are made by combining basic sacred form 805 with other sacred forms, such as sacred form 810, a three dimension tetrahedron with an octahedron within, as shown. By combining basic sacred form 805 with a plurality of other sacred forms, such as sacred form 810, large variation 820 of environment-affecting meditation tools preferably results. Additional embodiments will be shown in the figures to follow. The additional embodiments are preferably made by combining Sacred weaver form 1000, which is sanctioned as sacred by sanctioning church entity 148, with other sanctioned objects (sacred form 810) to create a large variation of environment-affecting sacred meditation tools (at least embodying herein a method, relating to environment-affecting meditation tools, comprising the steps of sanctioning as sacred, by at least one church group, at least one first set of objects; wherein the at least one first set of sacred objects consists essentially of environment-affecting objects; and sanctioning, by the at least one church group, use of the at least one first set of environment-affecting sacred objects to at least assist meditation; and sanctioning, by the at least one church group, specified combining of at least one second set of objects with at least one of the first set of environment-affecting sacred objects to create at least one third set of sacred objects, the third set of sacred objects comprising a large variation of environment-affecting sacred meditation tools).

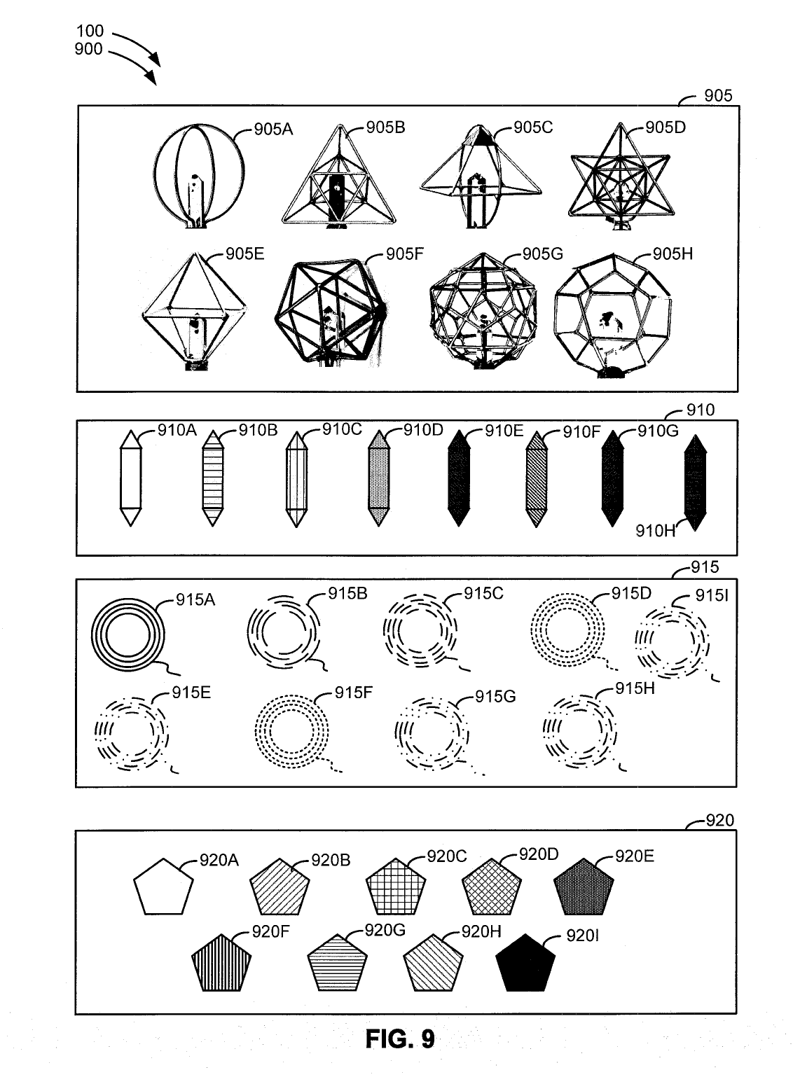

FIG. 9 shows several customer-selectable options of sacred objects and materials. Preferably, environment-affecting meditation tool system 100 comprises combinations of sacred objects with basic sacred form 805 shown in FIG. 8. FIG. 9 shows other shapes and characteristics considered sacred by applicant. Environment-affecting meditation tool system 100 preferably comprises sacred objects/characteristics 900, as shown.

Sacred objects/characteristics 900 may preferably comprise geometric structures 905, as shown. Geometric structures 905 preferably comprise shape 905A (preferably comprising arcs of a sphere), shape 905B (preferably comprising a tetrahedron with octahedron inside), shape 905C (preferably comprising a 51-degree pyramid with capstone), shape 905D (preferably comprising a star tetrahedron), shape 905E (preferably comprising an octahedron), shape 905F (preferably an icosahedron), shape 905G (preferably comprising a combined icosahedron and dodecahedron), and shape 905H (preferably comprising a dodecahedron), as shown.

Tools of environment-affecting meditation tool system 100 may preferably comprise crystal. Sacred objects/characteristics 900 preferably comprise color 910, as shown. Color 910 preferably comprises clear color 910A, or red color 910B, yellow/gold color 910C, green color 910D, blue color 910E, aqua color 910F, violet 910G, and multi-color 910H, as shown.

Tools of environment-affecting meditation tool system 100 may preferably comprise conductive wire material 915, as shown. Conductive wire material 915 preferably comprises copper wire 915A (preferably with titanium overwrap), titanium wire 915B, silver wire 915C, 12-Karat gold-filled wire 915D, 14-Karat white gold 915E, 14-karat yellow gold 915F, 18-Karat yellow gold wire 915G, and 22-Karat yellow gold wire 915H, and platinum wire 9151, as shown.

Tools of environment-affecting meditation tool system 100 may preferably comprise gemstone(s) 920, as shown. Gemstones 920 preferably comprise rubelite 920A (preferably red), tourmaline 920B (preferably green), topaz 920C (preferably yellow, preferably orange), quartz 920D (preferably violet, preferably clear), sapphire 920E, amethyst 920F, diamond 920G (preferably ½ Carat, ruby 920H, emerald 9201, as shown Upon reading the teachings of this specification, those of ordinary skill in the art will now understand that, under appropriate circumstances, other sacred objects/characteristics, such as, for example, various sizes, other colors, other geometric shapes, other wire materials, other gemstones, etc., may suffice.

Preferably, sacred objects/characteristics 900 are sanctioned by sanctioning church entity 148 (see FIG. 7), as shown. Preferably, sacred objects/characteristics 900 are selectable by customer 110 and incorporated into environment-affecting meditation tool (see FIGS. 1-6), as shown.

FIG. 10 shows a perspective view illustrating a first environment-affecting meditation tool form of the environment-affecting meditation tool system according to a preferred embodiment of the invention. Environment-affecting meditation tool system 100 preferably comprises first environment-affecting meditation tool form 1000, as shown. First environment-affecting meditation tool form 1000 is also referred to herein, for brevity purposes, as sacred weaver form 1000. Sacred weaver form 1000 is preferably used by a meditator in a meditation session. Sacred weaver form 1000 may be preferably held in the hands of the meditator or placed within the environment of the meditator. With reference to FIG. 8, sacred weaver form 1000 may also be attached or included as a component of other environment-affecting meditation tool forms as discussed. Sacred weaver form 1000 also is basic sacred form 805, the primary component, or building block, of environment-affecting meditation tool forms of environment-affecting meditation tool system 100.

A preferred structure of sacred weaver form 1000 is shown in FIG. 8. Sacred weaver form 1000 preferably comprises bar 1005, magnets 1010 (preferably two magnets), and conductive wire 1015, as shown. Preferably, sacred weaver form 1000 comprises the arrangement shown in FIG. 10. Conductive wire 1015 preferably comprises a metallic substance, preferably thin flexible wire of metal alloy. Sacred weaver form 1000 is preferably assembled in such a way that conductive wire 1015 wraps around bar 1005 to securely hold magnets 1010, under tension of conductive wire 1015, against bar 1005. Magnets 1010 are preferably secured against bar 1005 with conductive wire 1015 at about the center point of bar 1005, as shown. Proper positioning and placement of magnets 1010 is performed using a template tool comprising preferred measurement information and positioning information. Template tool preferably comprises outlines of bar 1005 and outlines where magnets 1010 should be positioned relative to bar 1005. Using template tool, magnets 1010 are preferably positioned and subsequently secured to bar 1005 with tape, preferably copper tape. Once magnets 1010 are secured with tape, wrapping of conductive wire 1015 around magnets 1010 and bar 1005 with tension is preferably performed. Conductive wire 1015 is preferably secured by wrapping around bar 1005 and magnets 1010 using wrapping pattern 1020, as shown.

Magnets 1010 are preferably arranged around bar 1005 so that a repulsive force is experienced between magnets 1010 (the magnetic poles of each magnet 1010 are aligned in the same direction). Magnet 1010 preferably comprises rare earth neodymium magnets having a magnetic energy product ranging from about 24 megagauss-oersted to about 54 megagauss-oersted. Magnet 1010 is preferably plated with 24-Karat gold.

In a highly preferred embodiment, the magnetic north pole of magnets 1010 is preferably positioned in a particular direction, preferably towards end portion 1025B of bar 1005. The pole orientation of magnets 1010 is preferably tested, preferably prior to assembly, with an electronic magnetic pole tester, preferably the electronic magnetic pole tester made commercially available by Industrial Magnetics, Inc., of Boyne City, Mich.

Bar 1005 preferably comprises terminating end portion 1025A, as shown. Bar 1005 is preferably double-terminated ending in end portion 1025A and end portion 1025B, as shown. End portion 1025A and end portion 1025B of bar 1005 are preferably 51-degree pyramids (the degree value is that value relating to the incline of the pyramid), and considered sacred by sanctioning church entity 148. Upon reading the teachings of this specification, those of ordinary skill in the art will now understand that, under appropriate circumstances, considering issues such as ability to position environment-affecting meditation tools end-to-end, etc., other geometries of terminal ends, such as flat, spherical, other polygonal, etc., may suffice.

Bar 1005 preferably comprises crystal. It is highly preferred that both ends of bar 1005 end in 51-degree pyramids at opposite ends of bar 1005 (i.e., the bar is “double terminated”), as shown. Bar 1005 is preferably laboratory-grown pure quartz, preferably Siberian quartz being a highly preferred type of such laboratory-grown pure quartz.

Bar 1005 may preferably be clear and translucent. Bar 1005 is preferably colorless. Bar 1005 may also preferably comprise at least one color of colors 910 (see FIG. 9). The following colors are preferred for bar 1005: red, orange, yellow, blue, green, aqua, violet, and multicolored (preferably achieved by adding colored gel(s) to bar 1005). The preferred colors of sacred weaver form 1000 are those color(s) that are sanctioned by sanctioning church entity 148, preferably colors 910. Preferably, sacred weaver form 1000 are sold individually or sold in a group, such group preferably comprising one sacred weaver form 1000 in each color.

With respect to the size dimensions of sacred weaver form 1000, a preferred length is about one inch (preferably for wearing and adorning) to about seven inches long (preferably for hand holding and adorning). A preferred the width of sacred weaver form 1000 is about one-quarter inch to about three-quarter inches. A preferred weight of sacred weaver form 1000 is about one and one-quarter ounce to about three and five-eighths ounces.

Sacred weaver form 1000 is preferably wrapped with conductive wire 1015 in a symmetric manner, as shown. The number of wraps used in sacred weaver form 1000 preferably varies based on the preferred dimensions of the sacred weaver form 1000 embodiment. For sacred weaver form 1000 having a preferred length of two and one-half inches, the following wrap patter is preferred (starting from end portion 1025A): seven wraps, followed by two wraps, followed by 19 wraps, followed by two wraps, followed by seven wraps (“ending” at end portion 1025B). For a sacred weaver form 1000 having a preferred length of about three and one half inches in length, the following wrap numbers are preferred (starting from end portion 1025A): seven wraps followed by two wraps, followed by 15 wraps, followed by two wraps, followed by seven wraps (“ending” at end portion 1025B). For a sacred weaver form 1000 having a preferred length of about seven inches, the following wrap numbers are preferred (starting from end portion 1025A): seven wraps, followed by two wraps, followed by 14 wraps, followed by two wraps, followed by seven wraps (“ending” at end portion 1025B). Upon reading the teachings of this specification, those of ordinary skill in the art will understand that, under appropriate circumstances, considering such issues as the length of the bar to be wrapped, other wrapping patterns and number of wrappings, may suffice.

It is highly preferred that a choice of conductive wire 1015 be provided for the benefit of a user customer so that the user customer may customize (via computer-mediated selective customer input) the appearance of sacred weaver form 1000. Preferably, a customer may customize first tool with a choice of material (preferably wire material 915, see FIG. 9) of conductive wire 1015, preferably sterling silver, 14-Karat yellow gold, 18-Karat yellow gold, 24-Karat yellow gold, 14-Karat white gold, platinum, copper, or titanium. Upon reading the teachings of this specification, those of ordinary skill in the art will now understand that, under appropriate circumstances, considering such issues as materials available, cost of materials, etc., other materials for conductive wire may suffice.

Sacred weaver form 1000 preferably comprises indicia 1030, as shown. Preferably, indicia 1030 comprises a geometric figure that is preferably sanctioned by sanctioning church entity 148. Indicia 1030 is preferably etched, preferably with a laser, onto surface of bar 1005. Preferably, geometric design 1030 is visible to a user of sacred weaver form 1000.

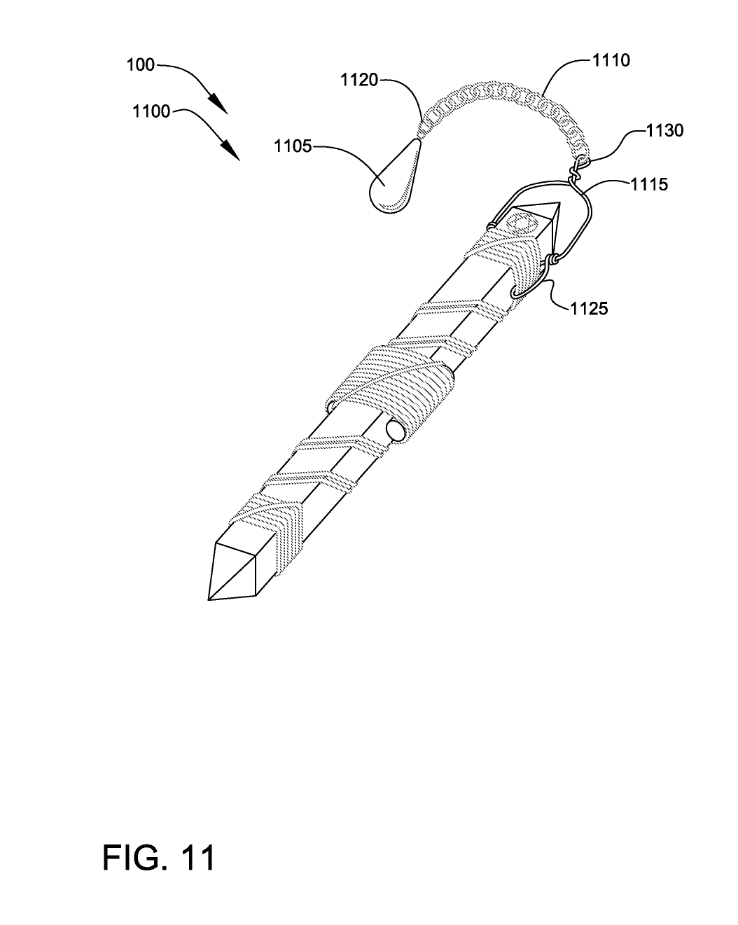

FIG. 11 shows a perspective view illustrating a second environment-affecting meditation tool form of the environment-affecting meditation tool system according to a preferred embodiment of the invention. Environment-affecting meditation tool system 100 preferably comprises second environment-affecting meditation tool form 1100 (for brevity purposes, sometimes referred to herein as Shambhala sacred weaver form 1100), as shown. Shambhala sacred weaver form 1100 is substantially similar to sacred weaver form 1000, as shown. Shambhala sacred weaver form 1100 further preferably comprises gripping element 1105, chain 1110, and chain attachment element 1115, as shown.

Gripping element 1105 is preferably attached to chain 1110 via split ring structure 1120, as shown. Gripping element 1105 preferably comprises crystal, preferably Siberian quartz crystal. Gripping element 1105 is preferably blue in color.

Chain attachment element 1115 is preferably of the same material as conductive wire 1015. Chain attachment element 1115 preferably comprises attachment loop 1125 that engages part of conductive wire 1015 and is positioned symmetrical about sacred weaver form 1000, as shown. Additionally, preferably, chain attachment element 1115 is preferably configured to extend over at least one end of sacred weaver form 1000 in a substantially “U” shaped structure, as shown. Preferably, chain attachment element 1115 is twisted into a second looping structure 1130, as shown. Chain 1110 is preferably connected to attachment loop 1130, as shown. Chain 1110 is preferably 24-Karat gold plated. Chain 1110 is preferably about four inches long.

Shambhala sacred weaver form 1100 is preferably used by a meditator by holding Shambhala sacred weaver form 1100 from gripping element 1105 with the fingers. It is highly preferred that such embodiment comprise split ring structure 1120; applicant has determined that such structure is the most robust and remains secured over a long period of time when used for typical meditation. A preferred shape of gripping element 1105 teardrop shaped, as shown. This preferred shape of gripping element 1105 compliments the curvature of the fingers to assist ease of use for meditative purposes.

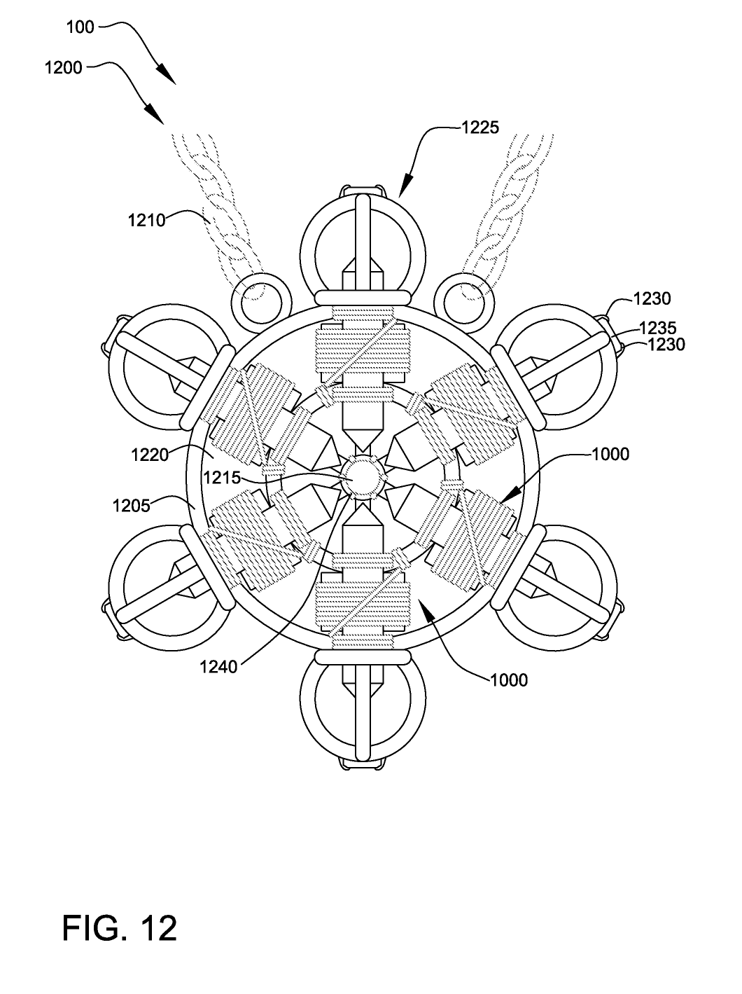

FIG. 12 shows a front plan view illustrating a third environment-affecting meditation tool form of the environment-affecting meditation tool system according to a preferred embodiment of the invention. Preferably, environment-affecting meditation tool system 100 comprises third environment-affecting meditation tool form 1200 (for brevity purposes, referred to as Shambhala radiator form 1200), as shown.

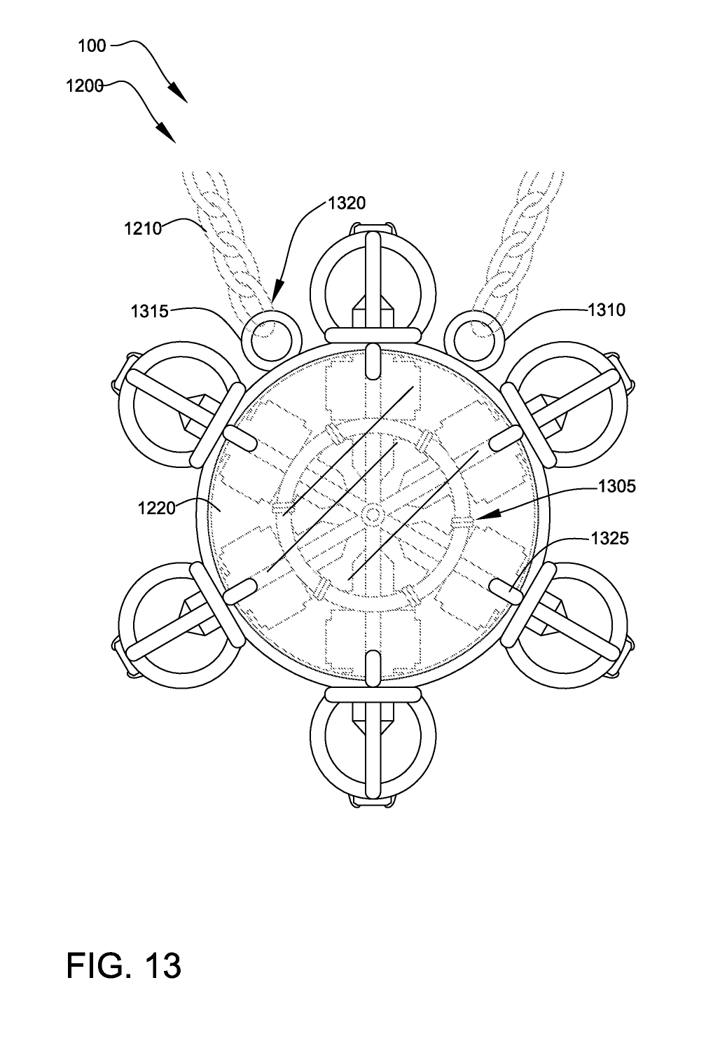

Preferably, Shambhala radiator form 1200 comprises at least two of sacred weaver form 1000, frame 1205, chain 1210, center gemstone 1215, and disc 1220 (more clearly shown in FIG. 13).

Preferably, at least two of sacred weaver form 1000 are attached to frame 1205, as shown. Preferably, between about four to about six of sacred weaver form 1000 are preferably securely attached to frame 1205 by wrapping conductive wire 1015 of sacred weaver form 1000 to frame 1205, as shown. Frame 1205 preferably comprises a framework structured and arranged to substantially support at least one bar of sacred weaver form 1000. Frame 1205 is preferably 24-Karat gold plated, preferably silver (preferably sterling silver), preferably titanium, preferably 18-Karat gold, preferably platinum, or preferably a material considered sacred (see FIG. 9).

Still additionally preferably, frame 1205 comprises the geometric configuration shown in FIGS. 12 and 13. Preferably, frame 1205 comprises at least geometric FIG. 1225. Preferably, geometric FIG. 1225 surrounds one end of each sacred weaver form 1000 incorporated into sacred weaver radiator form 1000, as shown. Preferably, geometric FIG. 1225 is metallic. Preferably, frame 1205 and geometric FIG. 1225 are preferably cast, preferably as a single piece. Preferably, material comprising frame 1205, and of geometric FIG. 1225, are preferably selectable by a customer from among sacred materials, preferably 24-Karat gold plated, sterling silver, titanium, 18-Karat gold, or platinum (see FIG. 9 and related discussion).

The shape of geometric FIG. 1225 shown in FIG. 12 is a preferred shape that is pre-sanctioned by sanctioning church entity 148. Preferably shape of geometric FIG. 1225 is customer selectable by a customer 110, as described herein, from among geometric structures 905 (see FIG. 9).

Preferably, at least two prongs 1230 extend away from geometric FIG. 1225 from its topmost portion, as shown. Prongs 1230 are positioned to securely hold in place gemstone 1235 over geometric FIG. 1225, as shown. Additionally, preferably, prongs 1230 may be selectable and customizable by customer 110 from among the following preferred materials: 24-Karat gold plated, sterling silver, titanium, 18-Karat gold, or platinum (see FIG. 9). Preferably, gemstone 1235 that is securely held by prong 1230 may be topaz, sapphire, clear quartz, blue Siberian quartz, violet Siberian quartz, amethyst, green tourmaline, red rubelite, sapphire, ruby, emerald, or diamond (see FIG. 9). Preferably, the material and gemstone 1235 are customer selectable by the methods described herein.

It is highly preferred that center gemstone 1215 be set in the center of frame 1205, as shown. Center gemstone 1215 is preferably securely held in place by at least four of prongs 1240, as shown. It is also highly preferred that the arrangement of prongs 1240 is such that the prongs do not spatially interfere with the alignment of the components of sacred weaver form 1000 or gemstone 1235, as shown. In other words, prongs 1240 are positioned such that they do not block or interfere with an end of sacred weaver form 1000, as shown. Applicant prefers such an arrangement of Shambhala radiator form 1200 as it preferably provides a meditation tool with a preferred meditative effect over other arrangements. Further the ability to select and customize an environment-affecting meditation tool (see FIG. 9 and related discussion) preferably adds to the preferred meditative affect. Preferably, center gemstone 1215 is selectable by a customer who may select among the following preferred gemstones: topaz, blue Siberian quartz, clears quartz, sapphire, and diamond.

FIG. 13 shows a back plan view of the embodiment of FIG. 12. Preferably, each sacred weaver form 1000 of Shambhala radiator form 1200 is preferably attached to frame 1205 with conductive wire 1015 wrapping at multiple attachment points, such as attachment point 1305, as shown.

Preferably, frame 1205 further comprises first small ring 1310 and second small ring 1315, as shown. Preferably, first small ring 1310 and second small ring 1315 are part of outermost edge of frame 1205, as shown. Chain 1210 preferably attaches to first small ring 1310 and second small ring 1315 via attachment 1320, as shown. Chain 1210 permits a meditator to hang or wear Shambhala radiator form 1200 as desired. Attachment 1320 is preferably a split ring structure, as shown, or alternately preferably a lobster claw attachment.

Shambhala radiator form 1200 further comprises prongs 1325 which assist in securely attaching disc 1220 preferably crystal, in position against frame 1205. Prongs 1325 are preferably located on frame 1205 and, generally, positioned beneath each sacred weaver form 1000. Crystal disc has a preferred diameter of about one and one-half inch. With respect to the material of disc 1220, quartz crystal is preferred. With respect to the color of disc 1220, the following colors are preferably selectable by customer 110 (according to the methods described herein): clear, aqua, blue, gold, or violet. Preferably, the aforementioned colors of disc 1220 are pre-sanctioned colors sanctioned, according to the methods described herein, by sanctioning church entity 148 (see FIG. 9 and related discussion).

Preferably, disc 1220 is etched, preferably laser etched, with a geometric design (not shown). Such geometric design show is preferably pre-sanctioned by sanctioning church entity 148. It is highly preferred that disc 1220 be etched as such etching enhances the association a meditator may have with sanctioning church entity 148 and bring about a more thorough meditation.

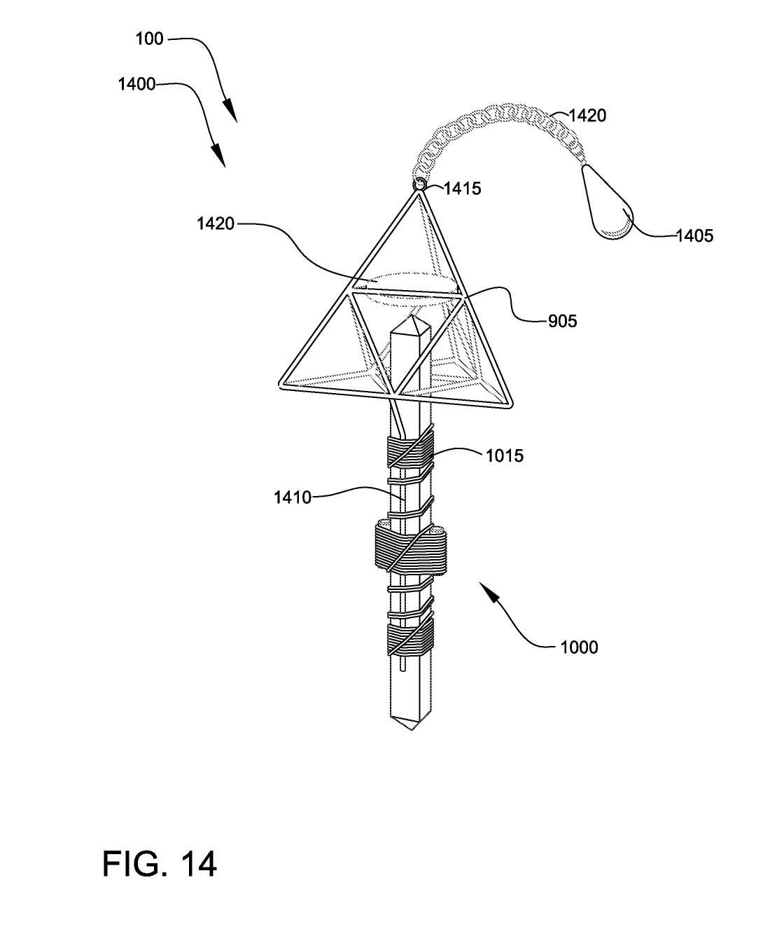

FIG. 14 shows a perspective view illustrating a fourth environment-affecting meditation tool form of the environment-affecting meditation tool system according to a preferred embodiment of the invention. Preferably, environment-affecting meditation tool system 100 comprises fourth environment-affecting meditation tool form 1400 (for brevity purposes, referred to as vajra sacred weaver form 1400), as shown.

Preferably, vajra sacred weaver form 1400 comprises sacred weaver form 1000, geometric structures 905, chain 1110, and gripping element 1405. A meditator preferably uses vajra sacred weaver form 1400 preferably by hand-holding the embodiment, by either sacred weaver form 1000 or gripping element 1405, during meditative sessions. Also, as shown, vajra sacred weaver form 1400 may be combined with other embodiments or used with other embodiments, as may be described herein, during meditation sessions.

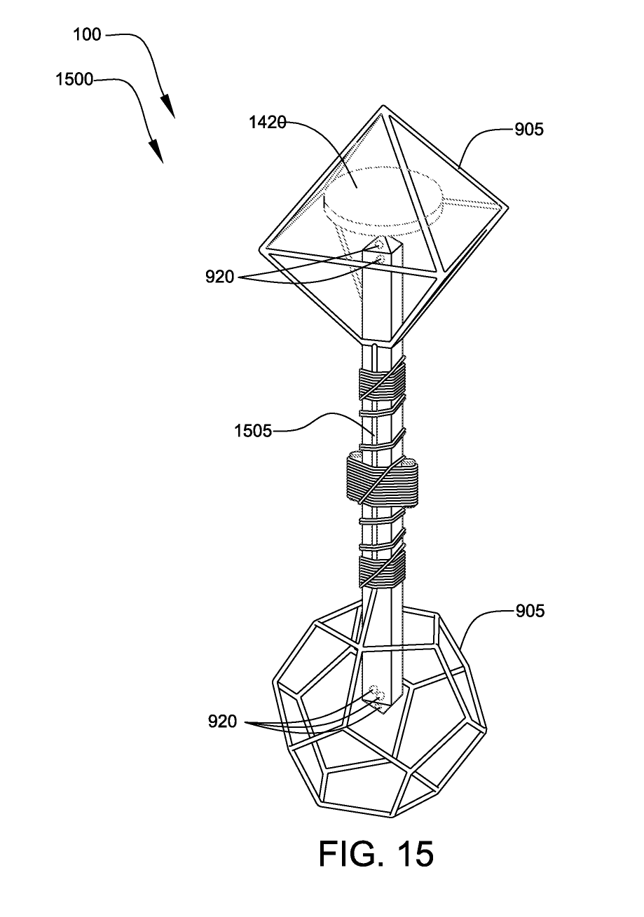

Preferably, geometric structure 905 surrounds at least one end portion of sacred weaver form 1000, as shown. Preferably, geometric structure 905 of vajra sacred weaver form 1400 is pre-sanctioned by sanctioning church entity 148. Preferably, any number or combination of geometric structure 905 may be used in vajra sacred weaver form 1400 in any manner that is considered sacred by sanctioning church entity 148 and a user of the embodiment (see FIG. 15 showing, preferably, two geometric structure 905 and FIG. 9).

Preferably, geometric structure 905 is secured to sacred weaver form 1000 by conductive wire 1015, as shown. Preferably, geometric structure 905 comprises elongated extension bar 1410, as shown. Elongated extension bar 1410 is preferably secured to sacred weaver form 1000 with conductive wire 1015, as shown. Elongated extension bar 1410 is preferably in a structural arrangement that is symmetrical with magnets of sacred weaver form 1000, as shown. Geometric structure 905 and elongated extension bar 141- are preferably welded in 3/32 silicon bronze and are preferably 24-Karat gold plated.

Geometric structure 905 of vajra sacred weaver form 1400 preferably comprises loop structure 1415 preferably on at least one end of geometric structure 905, as shown. Chain 1420 is preferably attached to loop structure 1415, as shown. Such attachment is preferably accomplished with a split ring structure. Further, gripping element 1405 is preferably attached to chain 1420, as shown. Such attachment, again, is preferably accomplished with a split ring structure, as shown.

Vajra sacred weaver form 1400 further preferably comprises element 1420 which is preferably a disc or Shambhala radiator form 1200. Under appropriate circumstances, other elements may be incorporated in the position of element 1420.

FIG. 15 shows a perspective view illustrating a fifth environment-affecting meditation tool form of the environment-affecting meditation tool system according to a preferred embodiment of the invention. Preferably, environment-affecting meditation tool system 100 comprises fifth environment-affecting meditation tool form 1500 (for brevity purposes, referred to as vajra form 1500), as shown.

Preferably, vajra form 1500 comprises sacred weaver form 1000, two of geometric structure 905, and gemstones 920, as shown. Geometric structures 905 are preferably 24-Karat gold plated cast bronze.

Preferably, geometric structures 905 are secured to sacred weaver form 1000, as shown. Geometric structures 905 are preferably connected by elongated extension bar 1505, as shown. Each geometric structure 905 of vajra form 1500 surround an “end” of sacred weaver form 1000, as shown.

Geometric structures 905 of vajra form 1500 may preferably be identical. In some instances, geometric structures 905 of vajra form 1500 are preferably not identical, as shown in FIG. 15. Preferably, geometric structures 905 of vajra form 1500 may be mixed and matched from the Geometric structures 905 shown in FIG. 9.

Gemstones 920 are preferably attached to sacred weaver form 1000 of vajra form 1500, as shown. Preferably, vajra form 1500 comprises gemstones 920 attached to end portions of sacred weaver form 1000, as shown. Preferably, one “end” of sacred weaver form 1000 comprises any combination of one or more of the following preferred gemstones 920: amber, amethyst, and turquoise. Additionally, at least one other “end” of sacred weaver form 1000 comprises any combination of one or more of the following preferred gemstones 920: garnet, lapis, moldivite, and ruby.

Additionally, Shambhala radiator form 1200 may be preferably optionally attached to geometric structure 905 and positioned where element 1420 is shown.

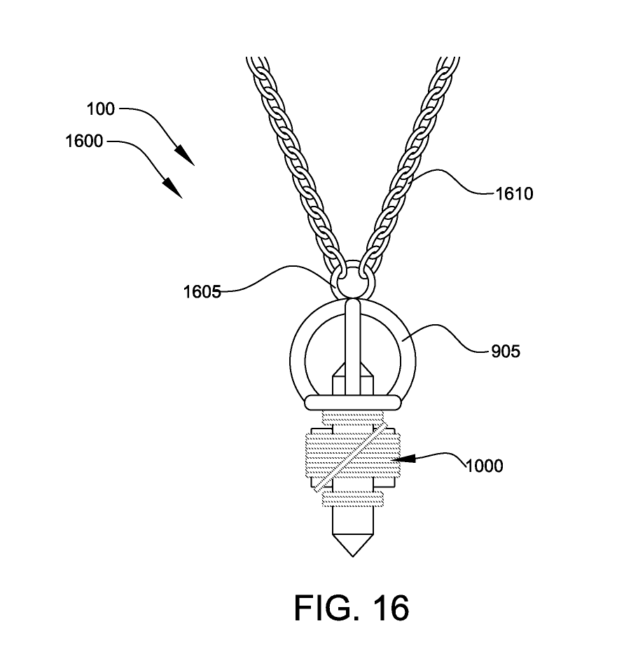

FIG. 16 shows a front view illustrating a sixth environment-affecting meditation tool form of the environment-affecting meditation tool system according to a preferred embodiment of the invention. Preferably, environment-affecting meditation tool system 100 comprises sixth environment-affecting meditation tool form 1600 (for brevity purposes, referred to as weaver pendant 1600), as shown. Weaver pendant 1600 preferably comprises sacred weaver form 1000, geometric structure 905, loop structure 1605, and chain 1610, as shown. Geometric structure 905 is preferably attached to sacred weaver form 1000 by wire wrapping with conductive wire 1015 of sacred waver form 1000.

Geometric structure 905 preferably comprises loop structure 1605 connected, preferably by welding, to at least one end of geometric structure 905, as shown. Loop structure 1605 may be preferably attached to chain 1610, as shown. In an alternate preferred embodiment, a post preferably replaces the chain so that weaver pendant 1600 may be worn as an earring, preferably as a set of earrings.

Geometric structure 905 is preferably positioned over an end of sacred weaver form 1000, as shown. In an alternate preferred embodiment, another geometric structure 905 may be positioned on the other end of sacred weaver form 1000. Geometric structure 905 is preferably one of the shapes pre-sanctioned by sanctioning church entity 148 (see FIG. 9). Geometric structures 905 of weaver pendant 1600 may preferably be identical (when weaver pendant 1600 comprises at least two geometric structure 905). Geometric structures 905 of weaver pendant 1600 may also preferably be different (preferably selections of geometric structures 905).

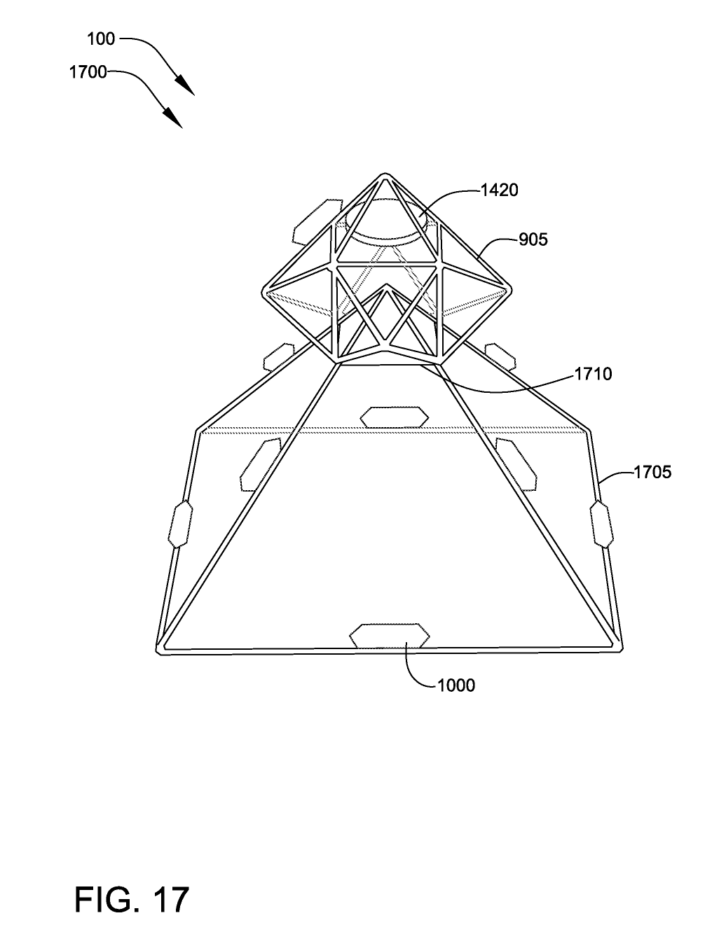

FIG. 17 shows a front perspective view illustrating a seventh environment-affecting meditation tool form of the environment-affecting meditation tool system according to a preferred embodiment of the invention. Preferably, environment-affecting meditation tool system 100 comprises seventh environment-affecting meditation tool form 1700 (for brevity purposes, referred to as head pyramid 1700), as shown. Head pyramid 1700 is preferably used by wearing head pyramid 1700 upon the head of a mediator during mediation sessions. Preferably, head pyramid 1700 comprises pyramid structure 1705, at least one sacred weaver form 1000, and capstone 1710, as shown. Any number or combination of sacred weaver form 1000 may be used with Head pyramid 1700. In one preferred embodiment, a plurality of sacred weaver form 1000 are aligned end to end along pyramid structure 1705, as shown.

Pyramid structure 1705 is preferably in the shape of a 51-degree pyramid (i.e., the angle of inclination of the pyramid sides is 51 degrees; also such shape being a sanctioned shape), as shown. Preferably, geometric structure 905 is attached to the top of pyramid structure 1705, as shown. Additionally, preferably, capstone 1710 may be preferably affixed to the top of pyramid structure 1705, as shown. Capstone 1710 may be preferably positioned substantially underneath the center of geometric structure 905, as shown. Paint may preferably be applied to the inner portion of capstone 1710.

Additionally, Shambhala radiator form 1200 may be preferably attached, in the position of element 1420, to second geometric shape 402, as shown. Also, at least one sacred weaver form 1000 may be suspended from at least one portion of geometric structure 905 preferably by a chain such that sacred weaver form 1000 preferably hangs preferably below the lowest edge of geometric structure 905.

Additionally, a plurality of head pyramid 1700 may be joined, preferably to be hung on a wall, to result in a combined embodiment preferably having the following preferred dimensions: about 18½-inches wide by about 18½-inches long by about 13-inches high; about 18½-inches wide by about 37-inches long, by about 13-inches high; and about 37-inches wide by about 37-inches long by about 13-inches high.

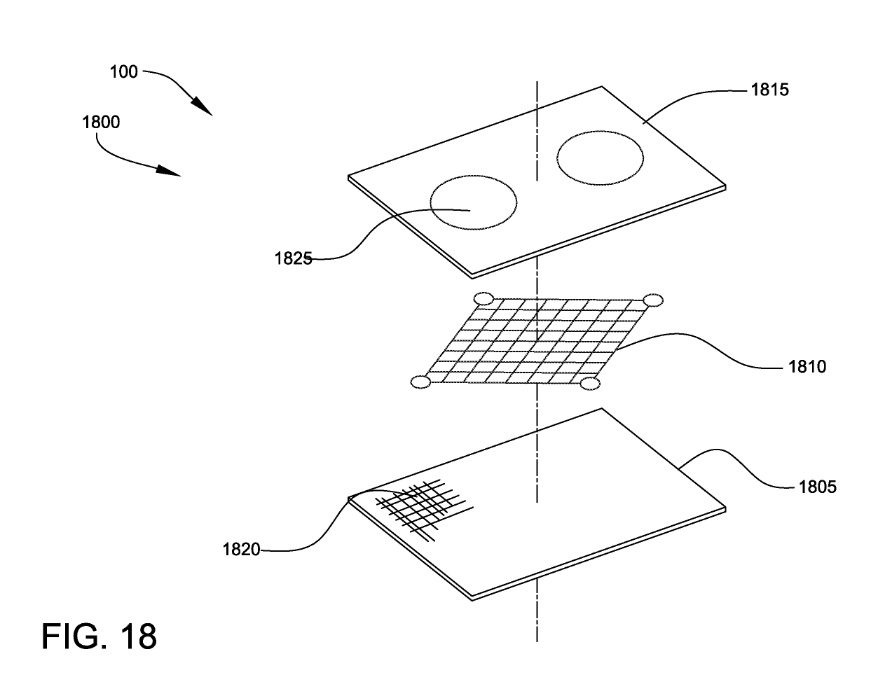

FIG. 18 shows an exploded view illustrating an eight environment-affecting meditation tool form of the environment-affecting meditation tool system according to a preferred embodiment of the invention. Preferably, environment-affecting meditation tool system 100 comprises eighth environment-affecting meditation tool form 1800 (for brevity purposes, referred to as metatron mat 1800), as shown. Metatron mat 1800 preferably comprises inner layer 1805, middle layer 1810, and outer layer 1815, and.

With respect to preferred dimensions of metatron mat 1800, one preferred embodiment of metatron mat 1800, with all layers assembled, comprises a length of about 30 inches and a width of about 18 inches. Also, metatron mat 1800 may preferably comprise a length of about five feet and a width of about 18 inches.

Preferably, middle layer 1810 is positioned substantially between inner layer 1805 and outer layer 1815. Additionally, preferably, a mesh-like structure 1820 is positioned substantially between inner layer 1805 and middle layer 1810.

Middle layer 1810 is preferably in the shape of a grid, as shown. Inner layer 1805 and outer layer 1815 are preferably made of cloth. Outer layer 1815 may be preferably adorned with at least one geometric FIG. 1825, as shown. Preferably, two of geometric FIGS. 1825 adorn outer layer 1815, as shown.

Preferably, geometric FIG. 1825 is among the figures pre-sanctioned by sanctioning church entity 148. Additionally, optionally, under appropriate circumstances, geometric figure(s) 1825 may be combined in any manner so long as such combination is sanctioned or sanctionable by sanctioning church entity 148.

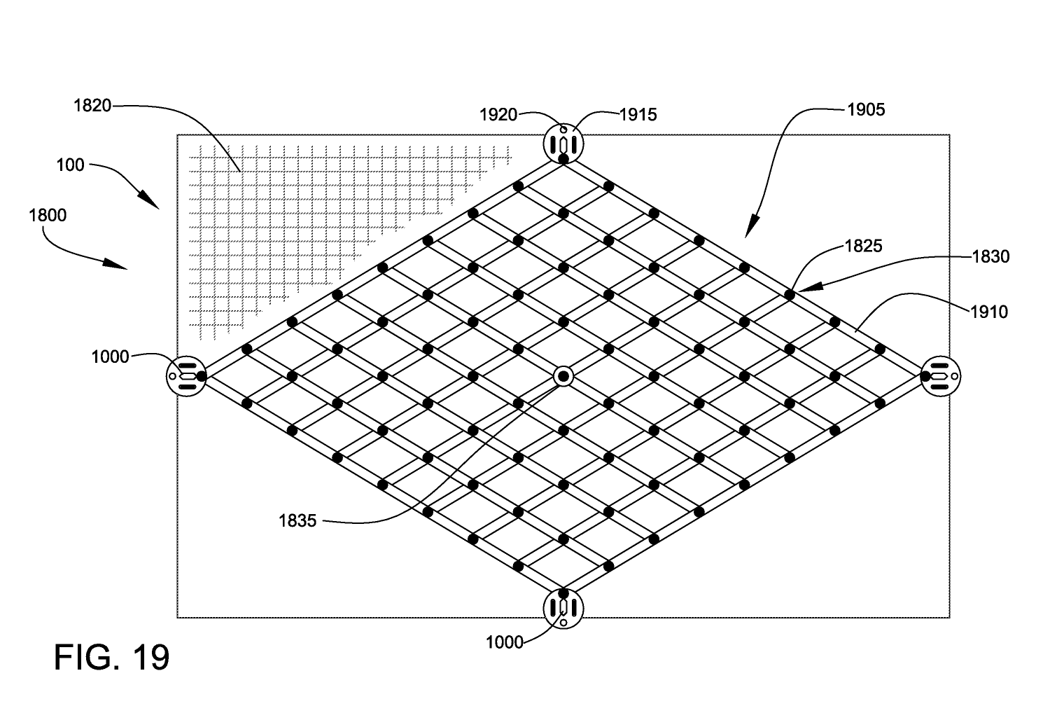

FIG. 19 shows a close up view of a middle layer of the embodiment of FIG. 18. Preferably, mesh-like structure 1820 is attached to inner layer 1805, as shown.

Preferably, middle layer 1810 preferably comprises tape 1910 (preferably fabric tape, preferably copper fabric tape) arranged as grid 1905, as shown. Additionally, grid 1905 has a longest length of about 30 inches and a preferred longest width of about 18 inches. Additionally, optionally, grid 1905 may be preferably about five feet in length and about 18 inches wide (at its widest point). Additionally, preferably, metatron mat 1800 may have one grid 1905, as shown. Optionally, under appropriate circumstances, metatron mat 1800 preferably comprises two of grid 1905. Grid 1905 is preferably rhombus shaped. Grid 1905 preferably comprises an arrangement of a plurality of tiled rhombi, as shown. Grid 1905 is preferably attached to mesh-like structure 1820. Preferably, at least one magnet 1825 is placed at each of the connecting points 1830 of grid 1905, as shown. Additionally, preferably, magnet 1825 may be preferably 24-Karat gold plated. Magnet 1825 is preferably a strong rare earth neodymium with a magnetic energy product ranging from about 24 megagauss-oersted to about 54 megagauss-oersted.

Additionally, grid 1905 preferably comprises disc 1915 at each terminating position of grid 1905, as shown. Each one of disc 1915 is placed on top of tape 1910, as shown. Additionally, preferably, disc 1915 comprises copper. Additionally, preferably, magnet 1825 is placed on top of disc 1915 over each terminating position of grid 1905, as shown Disc 1915 preferably comprise apertures 1920, as shown. Disc 1915 is connected to mesh-like structure 1820 via apertures 1920, as shown.

Preferably, at least one sacred weaver form 1000 is placed on first disc 1915. Sacred weaver form 1000 is preferably placed on disc 1915 between apertures 1920, as shown. Sacred weaver form 1000 is preferably placed on disc 1915 such that at least one part of sacred weaver form 1000 is in contact with magnet 1825, as shown.

Additionally, preferably, disc 1835 may be placed on fabric tape 1910 in the center of grid 1905, as shown. Additionally, preferably, disc 1835 may be copper. Additionally, preferably, a geometric design may be etched into disc 1835. Preferably, such geometric design is laser-etched. Additionally, preferably, the geometric figure may be among those pre-sanctioned by sanctioning church entity (see FIG. 9 and related discussion). Additionally, preferably, another disc, preferably of crystal, may be placed on top of disc 1835, as shown.

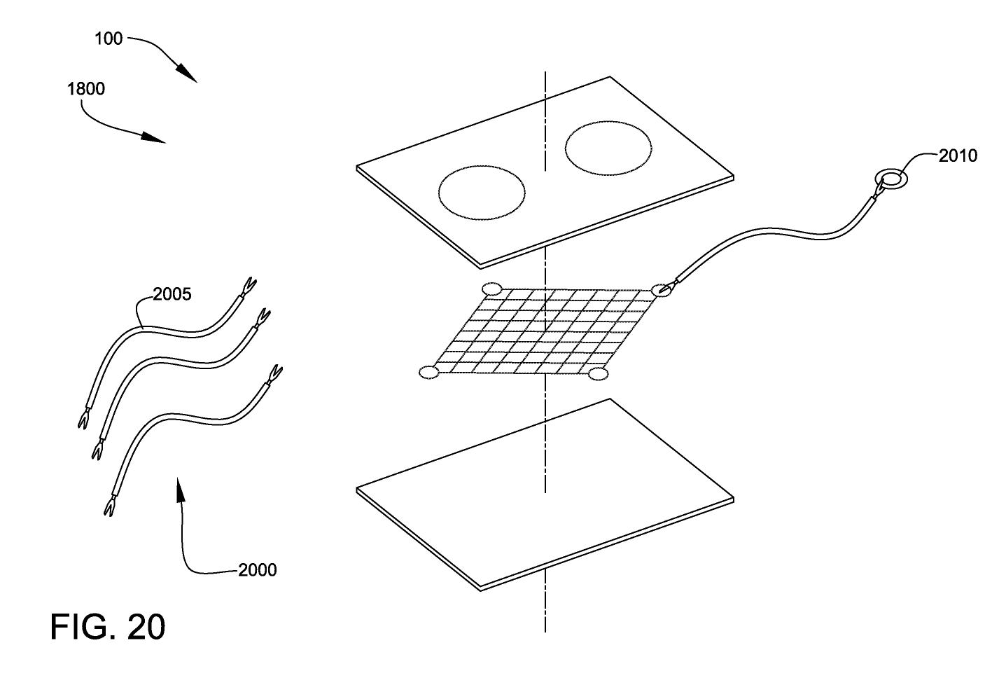

FIG. 20 shows additional components of the embodiment of FIG. 18. Preferably, apertures 1920 remains accessible when metatron mat 1800 is assembled. Metatron mat 1800 further comprises includes at least one wire set 2000 (preferably comprising at least four wires 2005), as shown. Additionally, preferably three of wire 2005 of wire set 2000 preferably comprises alligator clip connectors on one end of the wire 2005 and a male to female connector on the opposing end. Wires 2005 are preferably coupled with grid 1905 during use. Metatron mat 1800 is preferably positioned over a surface to be laid or say upon.

Additionally, optionally, under appropriate circumstances, plate 2010 may be connected to at least one wire in wire set 2000, as shown. Also, sacred weaver form 1000 (or other embodiments described herein) of environment-affecting tool system 100 may be attached to metatron mat 1800 with wire(s) 2005.

Additionally, preferably, plate 2010 has a preferred diameter of about 2 inches. Additionally, preferably, plate 2010 may be 24-Karat gold plated copper. Additionally, optionally, plate 2010 may have a geometric design etched into one or both sides. Additionally, optionally, under appropriate circumstances, plate 2010 may have a crystal disc placed upon it. Additionally, preferably, such crystal disc may have a geometric figure etched into it. Additionally, preferably, the geometric figure may be etched into crystal disc with a laser.

Additionally, optionally, under appropriate circumstances, plate 2010 may be used, for example, to hold essential oils, flora, homeopathic meditation tools, etc. or any combination thereof.

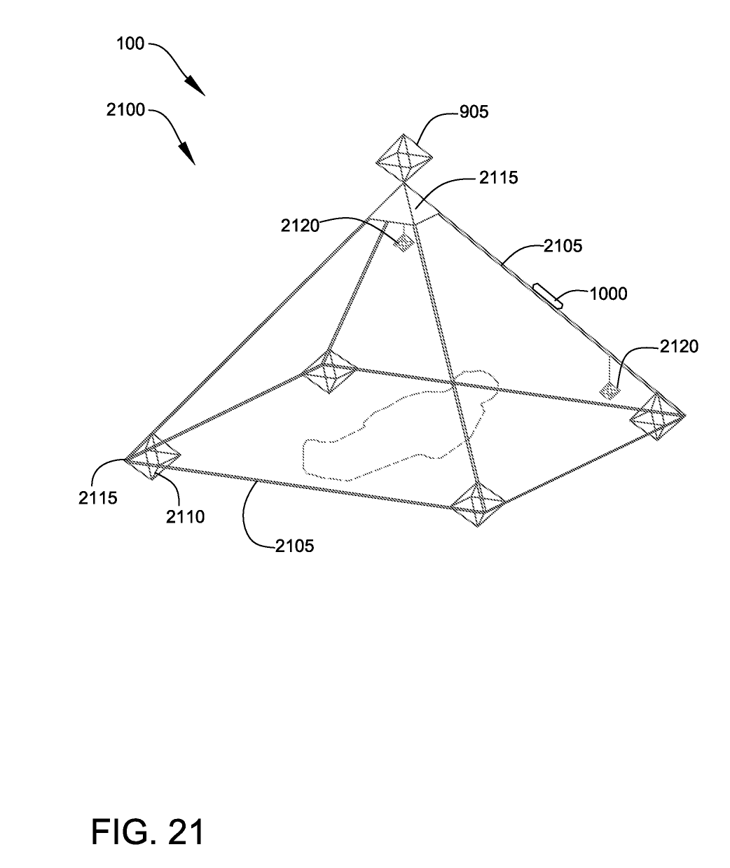

FIG. 21 shows a perspective view illustrating a ninth environment-affecting meditation tool form of the environment-affecting meditation tool system according to a preferred embodiment of the invention. Preferably, environment-affecting meditation tool system 100 comprises ninth environment-affecting meditation tool form 2100 (for brevity purposes, referred to as meditation pyramid 2100), as shown.

Preferably, meditation pyramid 2100 comprises poles 2105, corner piece 2110, capstone 2115, geometric structure 905, and at least one sacred weaver form 1000.

Preferably, poles 2105 are provided in a kit comprising at least eight of poles 2105. Poles 2105 are preferably copper; preferably coated with white enamel.

Preferably, poles 2105 (preferably three) fit into pole connector 2115 to form at least one corner of meditation pyramid 2100. Additionally, optionally, under appropriate circumstances, pole connector 2115 may be configured to fit into a plurality of pole(s) 2105.

Additionally, pole connector 2115 is an example of a geometric shape that is considered sacred and pre-sanctioned by sanctioning church entity 148. Additionally, preferably, under appropriate circumstances, pole connector 2115 may be substituted for a geometrically shaped connector among those considered sacred by the user. Additionally, under appropriate circumstances, any number of pole connector 2115 may be combined in any manner that is considered sacred by the user.

Additionally, optionally, under appropriate circumstances, meditation pyramid 2100 may be adjusted to a variety of preferred sizes by using a plurality of pole(s) 2105 of varying lengths. Additionally, optionally, under appropriate circumstances, at least three of poles 2105 slot together to form one “pole” to create meditation pyramid 2100 preferably having a base edge length of about seven feet. Additionally, two of poles 2105 slotted together to form one “pole” preferably create a meditation pyramid 2100 having preferred base edge length of about four feet and 10 inches. Additionally, optionally, one of pole 2105 preferably may be used to create meditation pyramid 2100 having a base length of about three feet and eight inches.

Additionally, optionally, under appropriate circumstances, at least one pole 2105 may be connected to additional poles 2105 in meditation pyramid 2100 to extend the dimensions of meditation pyramid 2100. Additionally, optionally, under appropriate circumstances, element 2120, i.e., any of the prior described embodiments of meditation tool system 100, may be connected to at least one pole 479. Preferably, meditation pyramid 2100 may be built to a preferred 51-degree angle.

The upper portion of meditation pyramid 2100 may preferably be capped with capstone 2115. Capstone 2100 preferably comprises 24-Karat gold plated, pure copper. Additionally, preferably, capstone 2100 may be at least about ten inches tall. Additionally, optionally, under appropriate circumstances, any embodiment of meditation tool system 100 may be symmetrically positioned at the midpoint of capstone 2100. Additionally, preferably, inside capstone 386 is a geometrically shaped corner connector that accommodates poles 2105 for securing purposes.

Additionally, optionally, under appropriate circumstances, geometrically shaped FIG. 905 (see FIG. 9) may be attached to capstone 2100.

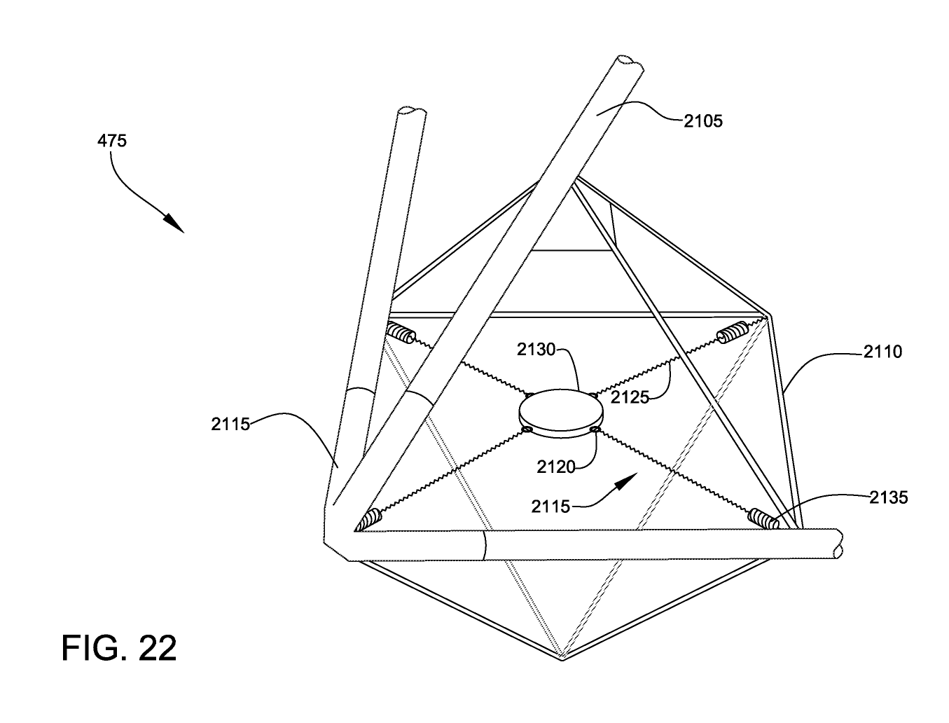

FIG. 22 shows a perspective view illustrating a corner piece system of the embodiment of FIG. 21. Corner piece system preferably comprises pole connector 2115, as shown. Poles 2105 preferably fit into pole connector 2115, as shown. Corner piece system further preferably comprises corner piece 2110, a geometric structure (see FIG. 9).

Preferably, an embodiment of environment-affecting meditation tool system 100 may be preferably suspended within geometrically corner piece 2110, preferably via attachment system 2115, as shown. Attachment system 2115 preferably comprises looping structure 2120 attached to at least one chain 2125, as shown. Preferably, looping structure 2120 is attached to at least one embodiment 2130 of environment-affecting meditation tool system 100 as shown. Additionally, preferably, chain 2120 may be attached to a spring structure 2135.

Upon reading the teachings of this specification, those of ordinary skill in the art will now understand that, under appropriate circumstances, other embodiments of environment-affecting tool system, such as combinations of the above-described embodiments, may suffice.

### PART III

To broaden the scope which sanctioning church entity 148 may communicate and serve the public, sanctioning church entity 148 preferably makes arrangements to deploy and offer church-related products and services related to sanctioning church entity 148 outside of the typical “church” environment. Preferably, services related to sanctioning church entity 148 may be offered to both church members and non-church-related individuals who may be guests of a spa, also sometimes referred to herein as spa guests.

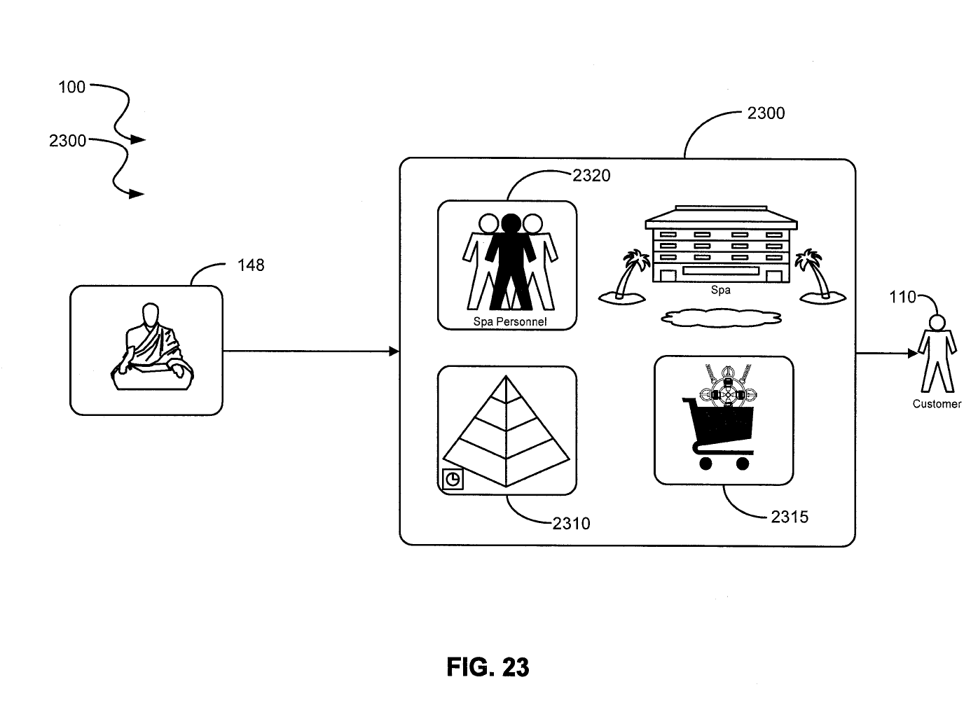

FIG. 23 shows a schematic diagram illustrating a preferred method of the present invention. Environment-affecting meditation tool system 100 preferably comprises method 2300, as shown.

In a preferred embodiment of the present invention sanctioning church entity 148 makes a business arrangement with spa 2305 so that spa 2305 may preferably offer services 2310 and goods 2315 related to sanctioning church entity 148. A preferred service 2310 related to sanctioning church entity 148 comprises offering meditation sessions within a cooperating spa, the meditation services being administered by spa personnel 2320 (a spa service provide) trained and certified by members of sanctioning church entity 148. Method 2300 at least embodies herein the step of sanctioning, by the at least one church group, training at least one other to provide church-group-sanctioned meditation services within at least one space, the space structured and arranged as specified by the at least one church group, the church-group-sanctioned meditation services using at least one of the first set of sacred objects consisting essentially of environment-affecting objects. Method 2300 further at least embodies herein providing, to such at least one trained spa service provider having at least one site, on-site retail distribution rights to sell the environment-affecting sacred meditation tools.

In a preferred embodiment of the present invention, a church related procedure preferably comprises a meditation session for an interval of time conducted in a particular room environment approved and sanctioned by sanctioning church entity 148. Such meditation session relates to placing a human individual who wishes and desires to receive the benefits of the meditation session into a particular room having a particular room environment approved by sanctioning church entity 148. Preferably, the walls of the room are decorated and adorned with art approved by sanctioning church entity 148. Preferably, the room comprises particular preferred furniture approved for use by sanctioning church entity 148 in particular preferred arrangements that maximize the meditative effect of church-related procedure.

Sanctioning church entity 148 has determined that it is highly preferred to place an article of furniture in a designated room. Such article of furniture preferably comprises a recliner seat. More preferably, article of furniture comprises a bed so that an individual may line prone during meditation session.

The article of furniture is preferably substantially beneath and within a pyramid system, as shown and described with respect to FIG. 21 and FIG. 22. The pyramid system preferably comprises a plurality of tubular elements arranged in a pyramidal geometric pattern.

To practice church-related procedure, a willing individual is preferably escorted into the room and instructed to sit or lie (depending on the article of furniture) on article of furniture. A spa personnel individual preferably escorts willing individual into room. Preferably, willing individual is provided, by spa 2305, a handheld environment-affecting meditation tool (see, e.g., FIG. 10, FIG. 11, and FIG. 15) to be held in the hands during the church-related procedure. Willing individual may also be provided with one or more wearable meditation tools (see, e.g., FIG. 12, FIG. 16, and FIG. 17). Certain other meditation tools, preferably sacred weaver form 1000 and others, are also preferably placed directly on the pyramid.

Preferably, music or sound effects preferably approved by sanctioning church entity 148, or more preferably recorded by sanctioning church entity 148, are audibly transmitted to a willing individual during a meditation session. Music or sound effects are either audibly transmitted to a willing individual with headphones or with loudspeakers.

Willing individual may make an offering to sanctioning church entity 148 prior to beginning meditation session.

Preferably, any doors of the room are closed to remove all unnecessary distractions from disturbing willing individual during meditation session.

The meditation session is preferably timed for an interval of time. Such interval of time may be provided in 30 minute increments. A willing individual may, for example, spend 30 minutes in a meditation session. Preferably, a timed meditation session will be paid for by a spa guest.

To establish the above meditation session as a regular, continuing service of a spa, in initial step, sanctioning church entity 148, or selected members of the sanctioning church entity 148 identify potential spas that may wish to offer church-related procedures to guests of the spa. Upon identifying a particular spa, by sanctioning church entity 148, to potentially offer the church-related therapy, a particular spa willing to cooperate with sanctioning church entity 148 must be selected. Several factors affect the selection by sanctioning church entity 148 of a willing-to-cooperate spa such as geographic location, reputation, spa-guest demographics, willingness to offer non-conventional spa services, etc. A spa that typically advertises to “new-agers”, those individuals accepting or living a “new-age” lifestyle, would be a preferred fit for sanctioning church entity 148. The term “new age” refers to an attitude of spiritual consciousness. Typical practices of “new-agers” include meditation, holistic medicine, vegetarianism, etc.

Once willing-to-cooperate spas are selected, a member (or members) of sanctioning church entity 148 preferably initiates communication or discussion with the selected willing-to-cooperate spas. Member of sanctioning church entity 148 will likely interact with a spa owner or spa manager. Preferably, member of sanctioning church entity 148 will provide a set of guidelines to willing-to-cooperate spas so that a willing-to-cooperate spa may determine whether that spa has the proper infrastructure to host and offer the church-related procedure. Sanctioning church entity 148 guidelines preferably include requirements that must be adhered to so that the willing-to-cooperate spa properly insures and certifies that the church related procedure will be performed in a proper and respectful manner so that spa guests achieve maximum benefit from the church-related procedure. Sanctioning church entity 148 guidelines may include details relating to the following: preferred room dimensions, preferred room temperature and/or humidity requirements, preferred room-entry and room-exit points, preferred room furniture (recliners, beds, pyramids, wall adornments), preferred room sound systems and speaker systems, etc. The willing-to-cooperate spa must determine whether or not that spa has the right space requirements, spa guest interest, spa personnel, etc., to host or offer the church-related procedure according to the guideline of sanctioning church entity 148. If the willing-to-cooperate spa does not have the proper infrastructure, the willing-to-cooperate spa may make a decision to pursue construction-related or other changes to accommodate the church related procedure guidelines. The willing-to-cooperate spa, likely through its management, must determine whether in an economic sense it can offer the church related procedure. The willing-to-cooperate spa that is confident it can handle sanctioning church entity 148 guidelines, preferably makes a commitment to offer the church-related procedure. The commitment is preferably memorialized with some type of writing, such as a contract. Subsequent to making the commitment, the now-committed spa will begin the next phase described below.

Sanctioning church entity 148 preferably provides materials necessary to use within a designated room within a spa. The spa will preferably provide a room with proper room dimensions by using an existing room, modifying an existing room, or constructing a new room as needed to comply with church related procedure guidelines.

After making a beneficial business arrangement, sanctioning church entity 148 may preferably agree to supply committed spa with the necessary components to provide church-related procedure/meditation session. Sanctioning church entity preferably agrees to provide necessary articles of furniture (e.g., recliner, bed, pyramid system, etc.), wall adornments, and meditation tools for use by spa guests. Either after supplying the above-mentioned articles, or contemporaneously, authorized members of sanctioning church entity 148 may begin training particular persons at the spa who will be administering church-related procedure.

With respect to training of spa personnel, authorized members of sanctioning church entity 148 will provide spa personnel with an introduction and history of the sanctioning church entity 148 and its church-related procedure. Preferably, authorized members of sanctioning church entity 148 provide written documentation, or more preferably video documentation, explaining proper administration of the church-related procedure. Subsequent to training program, the trainees and spa personnel should be able to perform the church-related procedure as required by the church.

Preferably, spa will use sanctioning church entity 148 for additional consulting. Spa may either enter into an ongoing consulting arrangement or provide further training arrangements with the church in the event that, for example, new spa-personnel hires or additional training must be performed to administer the church-related procedure. Alternately preferably, spa may have a member of sanctioning church entity 148 to administer church-related procedure on site.

Sanctioning church entity 148 preferably provides on-going training to the spa, either as church related therapy progresses, or as the tools needed to perform their church-related procedure change. As an illustration of this concept, if sanctioning church entity 148 determines that a later-developed pyramid system is preferred, the spa will receive training and materials to use the newly approved and certified pyramid system. The spa and spa-personnel may be certified and recertified if and when church-approved tools and methods evolve. In the event that a spa no longer wishes to provide the church related therapy, a business arrangement wherein the spa will return all sanctioning church entity 148 related therapy goods, and cease to provide the service will be carried out.

Spa may preferably advertise church-related procedure to the public by way of print advertisements, radio advertisements, television advertisements, Internet advertisements, travel agents, etc. Preferably the spa gets permission from the church to advertise the church related procedure in accordance with the rules of the sanctioning church entity. Spa guests will preferably register for the church-related procedure with the spa, not sanctioning church entity 148.

In addition to the above, sanctioning church entity 148 may preferably contract with spa to place a gift shop selling products (goods 2315) manufactured by sanctioning church entity 148 within the contracting spa. With a gift shop on site, a spa guest may purchase meditation tools or other meditation-related goods on site at the spa without having to visit a local chapter or boutique of sanctioning church entity 148 (which may be located in another state). Spa goers typically come from “out of town”.

Although applicant has described applicant's preferred embodiments of this invention, it will be understood that the broadest scope of this invention includes modifications. Such scope is limited only by the below claims as read in connection with the above specification. Further, many other advantages of applicant's invention will be apparent to those skilled in the art from the above descriptions and the below claims.

What is claimed is:

1) An environment-affecting meditation tool system comprising:
a) at least one bar having at least one terminal end;
b) at least one pair of magnets; and
c) at least one conductive wire;
d) wherein said at least one conductive wire attaches said at least one pair of magnets to said at least one bar by externally wire-wrapping said at least one pair of magnets; and
e) wherein each such magnet comprises a minimum magnetic energy product of about 24 megagauss-oersted;
f) wherein at least one immediate environment of said meditation tool is affected; and
g) wherein at least one particular feature of said environment-affecting meditation tool is custom-selectable by at least one user of said meditation tool from a variety of such custom-selectable features sacred to such user.

2) The environment-affecting meditation tool according to claim 1, wherein said at least one particular feature comprises said at least one conductive wire comprising sterling silver.

3) The environment-affecting meditation tool according to claim 1, wherein:
a) said at least one particular feature comprises said at least one conductive wire comprising sterling silver;
b) said sterling silver consists essentially of silver alloy; and
c) said silver alloy consists essentially of high purity silver.

4) The environment-affecting meditation tool according to claim 1, wherein said at least one particular feature comprises said at least one conductive wire comprising gold.

5) The environment-affecting meditation tool according to claim 1, wherein:
a) said at least one particular feature comprises said at least one conductive wire comprising gold;
b) said gold consists essentially of gold alloy; and
c) said gold alloy consists essentially of a minimum of 14 Karat purity.

6) The environment-affecting meditation tool according to claim 1, wherein said at least one particular feature comprises said at least one conductive wire comprising platinum.

7) The environment-affecting meditation tool according to claim 1, wherein:
a) said at least one particular feature comprises said at least one conductive wire comprising platinum;
b) said platinum consists essentially of platinum alloy; and
c) said platinum alloy consists essentially of high purity platinum.

8) The environment-affecting meditation tool according to claim 1, wherein said at least one particular feature comprises said at least one conductive wire comprising titanium.

9) The environment-affecting meditation tool according to claim 1, wherein:
a) said at least one particular feature comprises said at least one conductive wire comprising titanium;
b) said titanium consists essentially of titanium alloy; and
c) said titanium alloy consists essentially of high purity titanium.

10) The environment-affecting meditation tool according to claim 1, wherein said at least one particular feature comprises;
a) said at least one bar comprising crystal;
b) wherein said crystal consists essentially of quartz; and
c) wherein said quartz consists essentially of Siberian quartz.

11) The environment-affecting meditation tool according to claim 1, wherein said at least one particular feature comprises:
a) at least one indicia;
b) wherein said at least one indicia comprises an etching; and
c) wherein said at least one indicia is on said at least one terminal end of said at least one bar.

12) The environment-affecting meditation tool according to claim 1, wherein said at least one particular feature comprises:
a) at least one extender extending outwardly from adjacent said at least one terminal end of said at least one bar;
b) wherein said at least one extender comprises at least one chain and at least one handle; and
c) at least one split-ring configuration connecting said at least one chain to said at least one handle.

13) The environment-affecting meditation tool according to claim 1, wherein said at least one particular feature comprises:
a) said at least one extender comprises at least one geometric configuration;
b) wherein said at least one geometric configuration is attached with said at least one extender adjacent said at least one terminal end of said at least one bar; and
c) wherein said at least one chain is attached to said at least one geometric configuration attached with said at least one extender.

14) The environment-affecting meditation tool according to claim 1, further comprising:
a) at least one framework;
b) at least one gemstone; and
c) at least one disc.

15) The environment-affecting meditation tool according to claim 14, wherein said at least one particular feature comprises said at least one framework comprising gold.

16) The environment-affecting meditation tool according to claim 15, wherein:
a) said gold consists essentially of gold alloy; and
b) said gold alloy consists essentially of a minimum of 14 Karat purity.

17) The environment-affecting meditation tool according to claim 14, wherein said at least one particular feature comprises said at least one framework comprising titanium.

18) The environment-affecting meditation tool according to claim 17, wherein:
a) said titanium consists essentially of titanium alloy; and
b) said titanium alloy consists essentially of high purity titanium.

19) The environment-affecting meditation tool according to claim 14, wherein said at least one particular feature comprises said at least one framework comprising sterling silver.

20) The environment-affecting meditation tool according to claim 19, wherein:
a) said sterling silver consists essentially of silver alloy; and
b) said silver alloy consists essentially of high purity silver.

21) The environment-affecting meditation tool according to claim 14, wherein said at least one particular feature comprises said at least one framework comprising platinum.

22) The environment-affecting meditation tool according to claim 21, wherein:
a) said platinum consists essentially of platinum alloy; and
b) said platinum alloy consists essentially of high purity platinum.

23) The environment-affecting meditation tool according to claim 14, wherein said at least one particular feature comprises:
a) at least four of said at least one bar, each bar having at least one terminal end;
b) wherein said at least one framework supports said at least four bars.

24) The environment-affecting meditation tool according to claim 14, wherein said at least one particular feature comprises said at least one gemstone comprising turquoise.

25) The environment-affecting meditation tool according to claim 14, wherein said at least one particular feature comprises said at least one gemstone comprising diamond.

26) The environment-affecting meditation tool according to claim 25, wherein said diamond consists essentially of one-half carat total weight.

27) The environment-affecting meditation tool according to claim 14, wherein said at least one particular feature comprises said at least one gemstone comprising sapphire.

28) The environment-affecting meditation tool according to claim 14, wherein said at least one particular feature comprises said at least one gemstone comprising ruby.

29) The environment-affecting meditation tool according to claim 14, wherein said at least one particular feature comprises said at least one gemstone comprising topaz.

30) The environment-affecting meditation tool according to claim 14, wherein said at least one particular feature comprises said at least one gemstone comprising crystal.

31) The environment-affecting meditation tool according to claim 30, wherein:
a) said crystal consists essentially of quartz;
b) wherein said quartz consists essentially of Siberian quartz.

32) The environment-affecting meditation tool according to claim 14, wherein said at least one particular feature comprises:
a) at least one setting comprising at least four prongs;
b) wherein said at least four prongs are positioned substantially so as not to be congruous with said at least one terminal end of said at least one bar;
c) wherein said at least four prongs secure said at least one gemstone on said at least one framework; and
d) wherein said at least one setting is configured such that said at least one gemstone will not be at a elevation higher than said at least one framework.

33) The environment-affecting meditation tool according to claim 14, wherein said at least one particular feature comprises:
a) said at least one disc comprising crystal;
b) wherein said disc-crystal consists essentially of quartz; and
c) wherein said disc-crystal-quartz consists essentially of Siberian quartz.

34) An environment-affecting meditation tool system comprising:
a) at least one bar having at least one terminal end;
b) at least one pair of magnets; and
c) at least one conductive wire;
d) wherein said at least one conductive wire attaches said at least one pair of magnets to said at least one bar by externally wire-wrapping said at least one pair of magnets; and
e) wherein each of said at least one pair of magnets comprises a minimum energy product of about 24 megagauss-oersted;
f) wherein at least one immediate environment of said meditation tool is affected; and
g) wherein said at least one conductive wire of said environment-affecting meditation tool comprises gold.

35) An environment-affecting meditation tool system comprising:
a) at least one bar having at least one terminal end;
b) at least one pair of magnets; and
c) at least one conductive wire;
d) wherein said at least one conductive wire attaches said at least one pair of magnets to said at least one bar by externally wire-wrapping said at least one pair of magnets; and
e) wherein each of said at least one pair of magnets comprises a minimum energy product of about 24 megagauss-oersted;
f) wherein at least one immediate environment of said meditation tool is affected; and
g) wherein said at least one conductive wire of said environment-affecting meditation tool comprises silver.

36) An environment-affecting meditation tool system comprising:
a) at least one bar having at least one terminal end;
b) at least one pair of magnets; and
c) at least one conductive wire;
d) wherein said at least one conductive wire attaches said at least one pair of magnets to said at least one bar by externally wire-wrapping said at least one pair of magnets; and
e) wherein each of said at least one pair of magnets comprises a minimum energy product of about 24 megagauss-oersted;
f) wherein at least one immediate environment of said meditation tool is affected; and
g) wherein said at least one conductive wire of said environment-affecting meditation tool comprises platinum.

37) An environment-affecting meditation tool system comprising:
a) at least one bar having at least one terminal end;
b) at least one pair of magnets; and
c) at least one conductive wire;
d) wherein said at least one conductive wire attaches said at least one pair of magnets to said at least one bar by externally wire-wrapping said at least one pair of magnets; and
e) wherein each of said at least one pair of magnets comprises a minimum energy product of about 24 megagauss-oersted;
f) wherein at least one immediate environment of said meditation tool is affected; and
g) wherein said at least one conductive wire of said environment-affecting meditation tool comprises titanium.

38) An environment-affecting meditation tool system comprising:
a) at least one bar having at least one terminal end;
b) at least one pair of magnets;
c) at least one conductive wire; and
d) at least one fragment of sacred stone;
e) wherein said at least one fragment of sacred stone is attached to said at least one bar;
f) wherein said at least one conductive wire attaches said at least one pair of magnets to said at least one bar by externally wire-wrapping said at least one pair of magnets;
g) wherein each of said at least one pair of magnets comprises a minimum energy product of about 24 megagauss-oersted;
h) wherein at least one immediate environment of said meditation tool is affected; and
i) wherein said at least one conductive wire of said environment-affecting meditation tool comprises titanium; and
j) wherein said at least one fragment of sacred stone comprises moldivite.

39) A method, relating to environment-affecting meditation tools, comprising the steps of:
a) sanctioning as sacred, by at least one church group, at least one first set of objects;
b) wherein the at least one first set of sacred objects consists essentially of environment-affecting objects; and
c) sanctioning, by the at least one church group, use of the at least one first set of environment-affecting sacred objects to at least assist meditation; and
d) sanctioning, by the at least one church group, specified combining of at least one second set of objects with at least one of the first set of environment-affecting sacred objects to create at least one third set of sacred objects, the third set of sacred objects comprising a large variation of environment-affecting sacred meditation tools; and
e) permitting, by the at least one church group, respective purchasers of the at least one third set of sacred objects to respectively custom design at least one respective custom object, by use of at least one designated website of the at least one church group.

40) The method according to claim 39, further comprising the steps of:
a) providing receiving payment for the at least one respective custom object;
b) providing making, once sanctioned by the at least one church group, of the at least one respective custom object; and
c) providing fulfilling of the at least one respective custom object to the respective purchaser of such at least one respective custom object.

41) The method according to claim 39, further comprising:
a) sanctioning, by the at least one church group, training at least one other to provide church-group-sanctioned meditation services within at least one space, the space structured and arranged as specified by the at least one church group, the church-group-sanctioned meditation services using at least one of the first set of sacred objects consisting essentially of environment-affecting objects.

42) The method according to claim 41, wherein such at least one other so trained comprise at least one spa service provider.

43) The method according to claim 42, further comprising providing, to such at least one trained spa service provider having at least one site, on-site retail distribution rights to sell the environment-affecting sacred meditation tools.

44) The method according to claim 40, further comprising the steps of:
a) providing sanctioning, by the at least one church group, of the at least one respective custom object;
b) providing at least one customer purchasing interface for selecting at least one custom aspect for such at least one respective custom object and purchasing the at least one respective custom object;
c) providing at least one merchant-banking payment system so that the at least one customer can provide payment for such at least one respective custom object;
d) providing at least one assembling tracking system to track assembly of the at least one respective custom object; and
e) providing at least one fulfillment tracking system to track fulfillment of the purchase.

45) A computer-implemented method comprising the steps of:
a) providing at least one first customer interface, accessible over at least one computer network, wherein a customer may select at least one environment-affecting meditation-tool to be purchased;
b) providing at least one second customer interface, accessible over at least one computer network, wherein a customer may select at least one particular custom feature of the selected environment-affecting meditation-tool to be purchased;
c) processing, by at least one processor, custom input selections of the at least one customer; and
d) outputting at least one custom specification listing of the custom environment-affecting meditation-tool selected to be purchased by the at least one customer; and
e) storing such custom specifications of the custom environment-affecting meditation-tool.

46) The computer-implemented method according to claim 45, further comprising the steps of:
a) providing at least one customer purchase interface, accessible over at least one computer network, wherein a customer may input purchase-related information;
b) providing at least one purchasing processor to carry out a customer purchase transaction using such customer input purchase-related information;
c) outputting display of confirmation of a purchase to the at least one purchasing customer.

47) The computer-implemented method according to claim 46, further comprising the step of providing at least one purchase fulfillment tracking processor to track fulfillment-related data of a purchased at least one custom environment-affecting meditation tool.

48) The computer-implemented method according to claim 47, further comprising the step of providing at least one assembly fulfillment tracking processor to track assembly-related data of at least one custom environment-affecting meditation tool.

49) The computer-implemented method according to claim 48, further comprising the step of providing at least one custom environment-affecting meditation-tool delivery fulfillment tracking processor to track delivery-related data relating to a purchased at least one custom environment-affecting meditation tool.

50) The computer-implemented method according to claim 45, further comprising providing at least one real-time customer-messaging interface, accessible over at least one computer network, to permit real-time input of a customer message; and providing at least one customer messaging processor to process the customer message to be displayed on at least one other computer connected to the at least one computer network.
